SEQUENCE LISTING (3 sequences)

SEQ ID NO: 1 - DNA, 226 bp, synthetic construct
  source 1..226: mol_type = other DNA
ttcttctgccctcccgctcccaagaacaaagatatatttgcaaggaaggtctgcaggccctcaccagcggccgttagggaactcgtcccactcctgggtacggtagatgtaactctttggtctggaggcccagaagatgggacgtacatcttcctctcggcgcttggggtgggcgctgagagcccagggtaggggacgcctgggtgaggatggggacagagaattg

SEQ ID NO: 2 - DNA, 226 bp, synthetic construct
  source 1..226: mol_type = other DNA
ttcttctgccctcccgctcccaagaacaaagatgtatttgcaaggaaggtctgcaggccctcaccagcggccgttagggaactcgtcccactcctgggtacggtagatgtaactctttggtctggaggcccagaagatgggacgtacatcttcctctcggcgcttggggtgggcactgagagcccagggtaggggacgcctgggtgaggatggggacagagaattg

SEQ ID NO: 3 - DNA, 13 bp, synthetic construct
  source 1..13: mol_type = other DNA
tctacgaatctag

(19) United States
(12) Patent Application Publication
(10) Pub. No.: US 2023/0360733 A1
(43) Pub. Date: Nov. 9, 2023

(54) METHODS FOR DEEP ARTIFICIAL NEURAL NETWORKS FOR SIGNAL ERROR CORRECTION

(71) Applicant: Life Technologies Corporation, Carlsbad, CA (US)

(72) Inventors: Cheng-Zong BAI, San Francisco, CA (US); Eugene INGERMAN, San Francisco, CA (US); Alison LAI, San Diego, CA (US); Werner PUSCHITZ, San Marcos, CA (US); Chao WANG, Santa Clara, CA (US)

(21) Appl. No.: 18/312,663

(22) Filed: May 5, 2023

Related U.S. Application Data
(60) Provisional application No. 63/338,810, filed on May 5, 2022.
(60) Provisional application No. 63/338,703, filed on May 5, 2022.

(51) Int. Cl. G16B 40/10; G16B 30/00

(52) U.S. Cl. CPC G16B 40/10; G16B 30/00

(57) ABSTRACT

A method for correcting signal measurements comprises an artificial neural network (ANN). The ANN receives a plurality of signal measurements in a channel of an input layer. The ANN is applied to the signal measurements and produces a plurality of signal correction values. The signal correction values may be subtracted from the signal measurements to form corrected signal measurements. The corrected signal measurements may be provided to a base caller to produce a sequence of base calls. The ANN may comprise a convolutional neural network (CNN). The CNN may have a U-NET architecture that includes an encoder and a decoder. The U-NET may include a Convolutional Block Attention Module (CBAM). The CBAM may applied to the outputs of a last pooling layer of the encoder and provides refined feature maps to a first layer of the decoder. The input signal measurements may be generated by a nucleic acid sequencing instrument.

### CROSS-REFERENCE TO RELATED APPLICATIONS

This application claims the benefit under 35 U.S.C. § 119(e) of U.S. Provisional Application No. 63/338,703, filed May 5, 2022, and U.S. Provisional Application No. U.S. 63/338,810, filed May 5, 2022. The entire contents of the aforementioned applications are incorporated by reference herein.

### SEQUENCE LISTING

The instant application contains a Sequence Listing which has been submitted electronically in XML format and is hereby incorporated by reference in its entirety. Said XML copy, created on April 27, 2023, is named TP109360USUTL1_SL.xml and is 3,922 bytes in size.

### FIELD OF THE INVENTION

This application generally relates to methods, systems, and computer-readable media for applying a deep learning artificial neural network for correction of signal data obtained by next-generation sequencing systems, and, more specifically, to correct the signal data for improving the accuracy of base calling.

### BRIEF DESCRIPTION OF THE DRAWINGS

FIG. 1A is a schematic diagram illustrating aspects of sequencing by synthesis in next generation sequencing. Figure discloses SEQ ID NOS 3 and 3, respectively, in order of appearance.

FIG. 1B shows an example of signal measurements and corresponding base calls.

FIG. 2A is an example illustrating principles of the base calling operations.

FIG. 2B shows an example of signal measurement distributions when the signal measurements have additive white Gaussian noise and no phasing error.

FIG. 3A shows an example of an ideal distribution of signal measurements with no shift due to systematic error.

FIG. 3B shows an example of a shift in the distribution of signal measurements due to systematic error.

FIG. 4 is an example of a block diagram of an analysis pipeline for signal data obtained from a nucleic acid sequencing instrument.

FIG. 5 is an example of a block diagram of an analysis pipeline including an artificial neural network for correcting signal data obtained from a nucleic acid sequencing instrument.

FIG. 6 illustrates an example of a CNN having a U-Net architecture and including a CBAM.

FIG. 7 is a table illustrating example improvements in base error rates.

FIG. 8 is an example of a block diagram of a labeling and training pipeline including an artificial neural network for correcting signal data obtained from a nucleic acid sequencing instrument.

FIG. 9 is an example of a block diagram a labeling pipeline.

FIG. 10 is an example diagram of a set of measured sequences.

FIG. 11 is an example of a block diagram of a method for labeling measurements.

FIG. 12 is an example of a block diagram of a method for generating sequences.

FIG. 13 is an example of a block diagram of a method for labeling sequences.

FIG. 14 is an example of a block diagram of a method for labeling read sequences.

FIG. 15 is an example of a block diagram for determining statistical characteristics for a group of measured sequences.

FIG. 16 is an example of a block diagram for training an artificial neural network.

### DETAILED DESCRIPTION

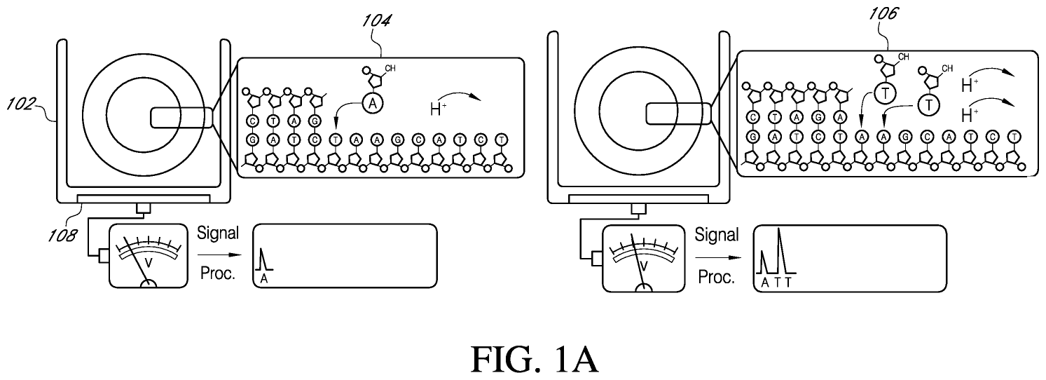

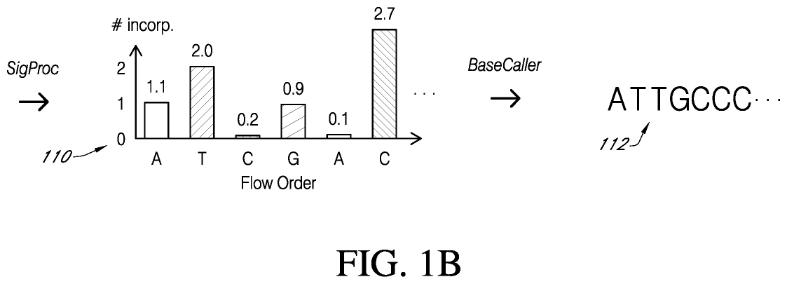

FIG. 1A is a schematic diagram illustrating aspects of sequencing by synthesis in next generation sequencing. For example, a well 102 is one of a plurality of reaction spaces on a microarray of the nucleic acid sequencing instrument. The well 102 contains a bead to which a population of template strands of nucleic acids are attached. Nucleotides are flowed over the wells one per flow in a flow order, such as A, T, C, G, A, C. The incorporation of a nucleotide complementary to the template sequence releases hydrogen ions, as illustrated in diagram 104 for the incorporation of a single nucleotide and diagram 106 for the incorporation of two nucleotides. The resulting change in pH is measured by a sensor 108, e.g., an ion-sensitive field effect transistor (ISFET). The raw data from the sensor includes background signal, incorporation signal and noise. Signal processing applied to the raw data from the sensor converts the raw data into signal measurements where the amplitudes are related to the number of incorporations for the particular nucleotide flowed. FIG. 1B shows an example of the signal measurements and the corresponding base calls. Graph 110 shows signal measurements determined from the signal processing applied to the raw data. The signal measurements are plotted versus the nucleotides of the flow order in graph 110. The signal measurements determined in response to flows of nucleotides in the flow order are also referred to as signal measurements in flow space or flow measurements. Applying a base caller to the signal measurements produces a sequence of bases, or sequence read 112. The purpose of the base caller is to determine the most likely base sequence that would correspond to the signal measurements. The number of nucleotide bases incorporated can be inferred by rounding to the nearest integer shown in the y-axis. In this example, the signal measurement corresponding to the flow of nucleotide A is 1.1, corresponding to a single base A in the sequence read 112. The signal measurement of 2.0 corresponds to two incorporations of the nucleotide T and the base call of the 2-mer TT in the sequence read 112. The signal measurement of 0.2 indicates no incorporation of nucleotide C. The signal measurement corresponding to the flow of nucleotide G is 0.9, corresponding to a single base G in the sequence read 112. The signal measurement of 0.1 indicates no incorporation of nucleotide A. The signal measurement of 2.7 corresponds to three incorporations of the nucleotide C and the base call of the 3-mer CCC in the sequence read 112.

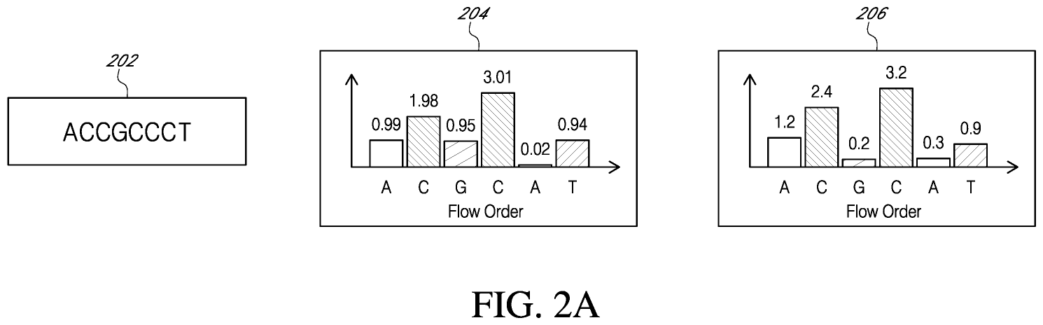

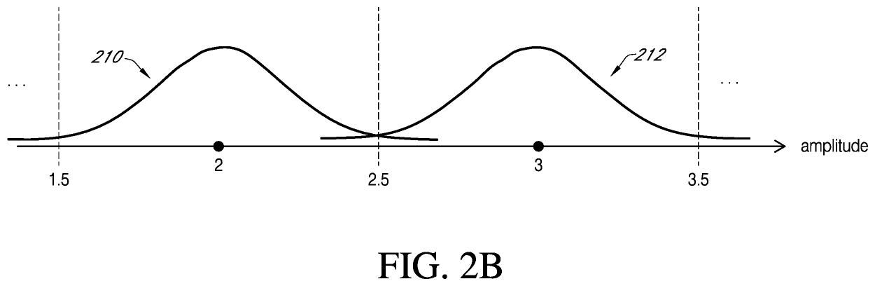

FIG. 2A is an example illustrating principles of the base calling operations. The goal of the base caller is to determine the base sequence 202 for which an expected signal 204 is most likely to be the signal measurements 206. The base caller may use a simulation model to generate simulated signal measurements, or expected signal 204. For example, generating the simulated signal measurements may include one or more features described in U.S. Pat. Appl. Publ. No. 2012/0109598 published May 3, 2012, incorporated by reference herein in its entirety. In some cases, the model is a phase-state model that simulates a population of template strands as it undergoes the sequencing process and becomes divided into different phase-states as the sequencing-by-synthesis progresses. The number of template strands in each phase-state may be calculated using one or more phasing effect parameters, such as the incomplete extension rate and/or the carry forward rate. The inputs to simulation model may include the signal measurements, a preliminary base sequence, nucleotide flow order and initial model parameters. The simulated signal measurements may be compared to the signal measurements to optimize the model parameters. For example, a simulation model can generate simulated signal measurements that would be expected if there were nucleotide incorporations, or no incorporation in the next flow. This can be done for a series including 0-mer, 1-mer, 2-mer, and so on, in order to generate an array of predicted signal measurements. These model-predicted signals can be compared against the actually measured signals to fit the model to the measured signal data.

The simulated signal measurements may be generated from a simulation model that includes model parameters such as a “carry forward” parameter and an “incomplete extension” parameter. One cause of phase synchrony loss is the failure of a sequencing reaction to incorporate one or more nucleotide species on a template strand for a given flow cycle, which may result in that template strand being behind the main template population in sequence position. This effect is referred to as an “incomplete extension” error. Another cause of phase synchrony loss is the improper incorporation of one or more nucleotide species on a template strand, which may result in that template strand being ahead of the main population in sequence position. This is referred to as a “carry forward” error. Carry forward errors may result from the misincorporation of a nucleotide species, or in certain instances, where there is incomplete removal of a previous nucleotide species in a reaction well (e.g. incomplete washing of the reaction well). Thus, as a result of a given flow cycle, the population of template strands may be a mixture of strands in different phase-states.

FIG. 2B shows an example of signal measurement distributions when the signal measurements have additive white Gaussian noise and no phasing error. Plot 210 shows an ideal distribution of amplitudes of signal measurements given the underlying true homopolymer is a 2-mer. Plot 212 shows an ideal distribution of amplitudes of signal measurements given the underlying true homopolymer is a 3-mer. The decision boundaries are shown by the dotted lines. For example, a 2-mer is called for amplitudes between 1.5 and 2.5, and a 3-mer is called for amplitudes between 2.5 and 3.5. A maximum likelihood decision provides an optimal approach that minimizes mean squared error for signals with additive white Gaussian noise. Gaussian noise adds unpredictable random error.

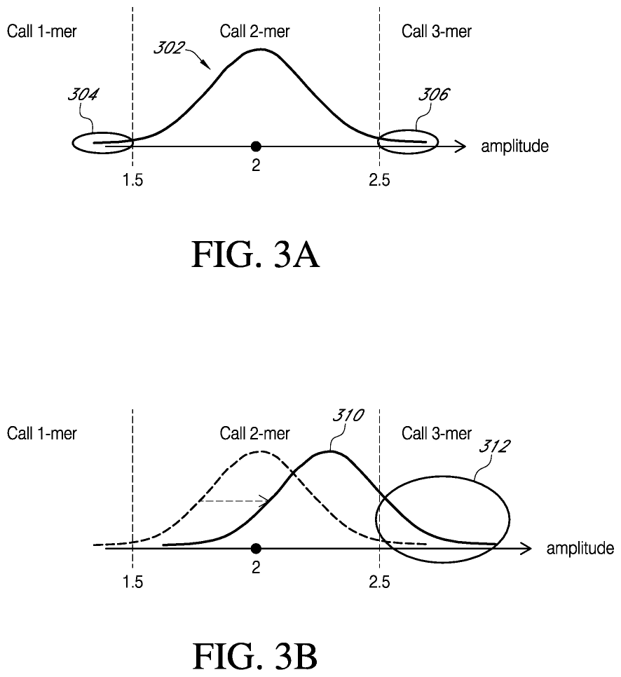

However, it has been observed from real data that a significant portion of error events are “systematic” and not random. Even low homopolymers with low noise variance may be affected by systematic error. Unlike random error, systematic error can be reproduced in different experiments. However, the origin of the systematic error may be complex and/or unknown. Systematic error may cause a shift in the distribution of signal measurements, such that the signal measurements may include noise with a non-zero mean. FIG. 3A shows an example of an ideal distribution of signal measurements with no shift due to systematic error. The plot 302 shows an ideal distribution of signal measurements given a 2-mer. The error populations 304 and 306 are outside the boundaries for calling a 2-mer. FIG. 3B shows an example of a shift in the distribution of signal measurements due to systematic error. The plot 310 of the distribution of signal measurements is shifted in the positive direction. The shift causes an increase in erroneous 3-mer calls which should be 2-mer calls, shown by the error population 312.

Aspects of embodiments of the present disclosure apply an artificial neural network to mitigate the effects of systematic error in the signal measurements and improve the accuracy of base calling.

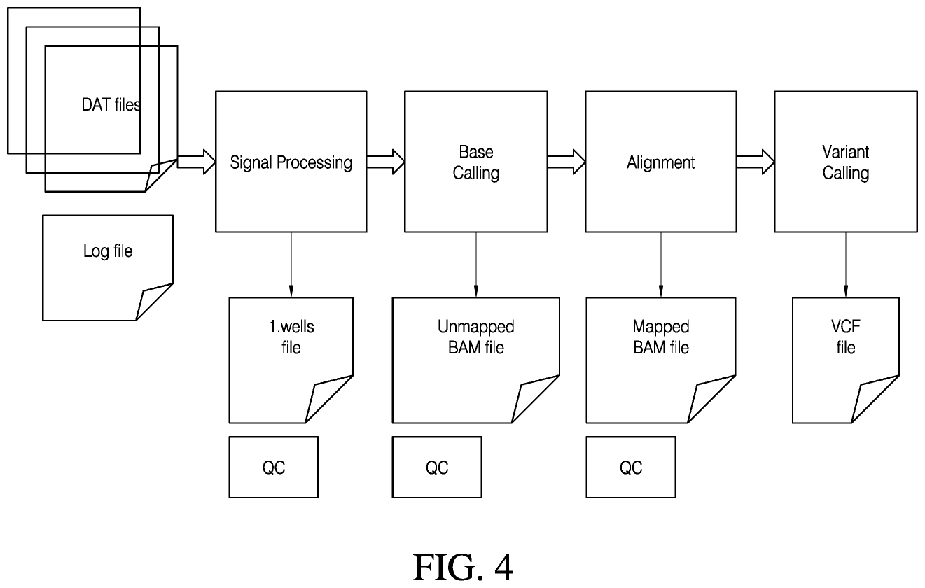

FIG. 4 is a block diagram of an analysis pipeline for signal data obtained from a nucleic acid sequencing instrument. The sequencing instrument generates raw data files (DAT, or .dat, files) during a sequencing run for an assay. Signal processing may be applied to raw data to generate incorporation signal measurement data for files, such as the 1.wells files, which are transferred to the server FTP location along with the log information of the run. The signal processing step may derive background signals corresponding to wells. The background signals may be subtracted from the measured signals for the corresponding wells. The remaining signals may be fit by an incorporation signal model to estimate the incorporation at each nucleotide flow for each well. The output from the above signal processing is a signal measurement per well and per flow, that may be stored in a file, such as a 1.wells file.

In some embodiments, the base calling step may perform phase estimations, normalization, and runs a solver algorithm to identify best partial sequence fit and make base calls. The base sequences for the sequence reads are stored in unmapped BAM files. The base calling step may generate total number of reads, total number of bases, and average read length as quality control (QC) measures to indicate the base call quality. The base calls may be made by analyzing any suitable signal characteristics (e.g., signal amplitude or intensity). The signal processing and base calling for use with the present teachings may include one or more features described in U.S. Pat. Appl. Publ. No. 2013/0090860 published Apr. 11, 2013, U.S. Pat. Appl. Publ. No. 2014/0051584 published Feb. 20, 2014, and U.S. Pat. Appl. Publ. No. 2012/0109598 published May 3, 2012, each incorporated by reference herein in its entirety.

Once the base sequence for the sequence read is determined, the sequence reads may be provided to the alignment step, for example, in an unmapped BAM file. The alignment step maps the sequence reads to a reference genome to determine aligned sequence reads and associated mapping quality parameters. The alignment step may generate a percent of mappable reads as QC measure to indicate alignment quality. The alignment results may be stored in a mapped BAM file. Methods for aligning sequence reads for use with the present teachings may include one or more features described in U.S. Pat. Appl. Publ. No. 2012/0197623, published Aug. 2, 2012, incorporated by reference herein in its entirety.

The BAM file format structure is described in “Sequence Alignment/Map Format Specification,” Sep. 12, 2014 (github.com/samtools/hts-specs). As described herein, a “BAM file” refers to a file compatible with the BAM format. As described herein, an “unmapped” BAM file refers to a BAM file that does not contain aligned sequence read information and mapping quality parameters and a “mapped” BAM file refers to a BAM file that contains aligned sequence read information and mapping quality parameters.

The variant calling step may include detecting single-nucleotide polymorphisms (SNPs), insertions and deletions (InDels), multi-nucleotide polymorphisms (MNPs), and complex block substitution events. In various embodiments, a variant caller can be configured to communicate variants called for a sample genome as a *.vcf, *.gff, or *.hdf data file. The called variant information can be communicated using any file format as long as the called variant information can be parsed and/or extracted for analysis. The variant detection methods for use with the present teachings may include one or more features described in U.S. Pat. Appl. Publ. No. 2013/0345066, published Dec. 26, 2013, U.S. Pat. Appl. Publ. No. 2014/0296080, published Oct. 2, 2014, and U.S. Pat. Appl. Publ. No. 2014/0052381, published Feb. 20, 2014, and U.S. Pat. No. 9,953,130 issued Apr. 24, 2018, each of which is incorporated by reference herein in its entirety. In some embodiments, the variant calling step may be applied to molecular tagged nucleic acid sequence data. Variant detection methods for molecular tagged nucleic acid sequence data may include one or more features described in U.S. Pat. Appl. Publ. No. 2018/0336316, published Nov. 22, 2018, incorporated by reference herein in its entirety.

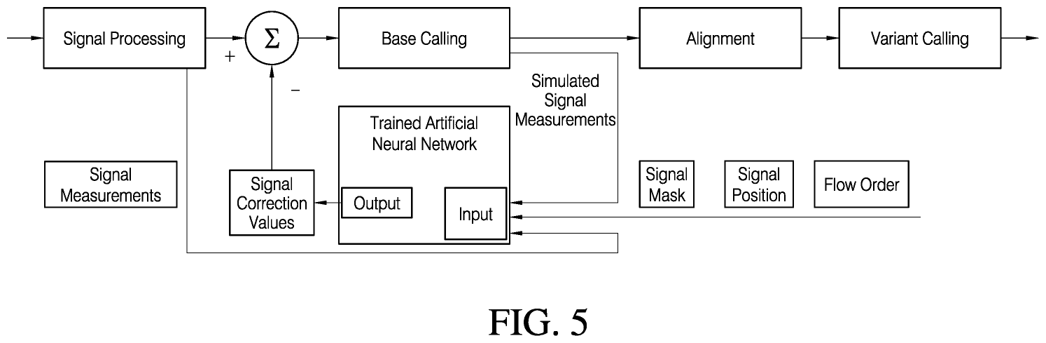

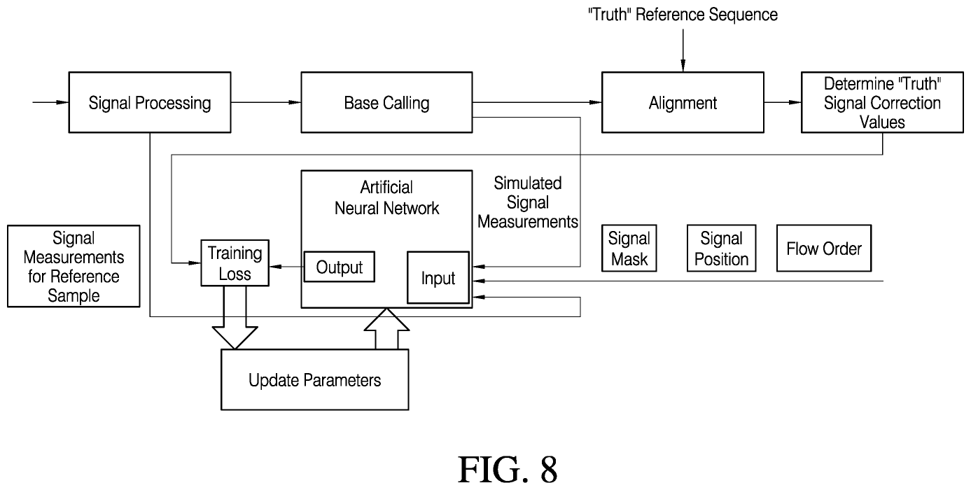

An artificial neural network (ANN) may operate in a training mode and an inference mode. During the training mode, the ANN receives training data from the input channels. The training data corresponds to a truth set of base calls. The ANN adapts its parameters in accordance with a loss function, as described below with respect to FIG. 8. During inference mode, as described below with respect to FIG. 5, the ANN receives data from the input channels resulting from signal processing applied to raw data from a sequencing run by the nucleic acid sequencing instrument. The parameters of the ANN have fixed values during the inference mode.

FIG. 5 is a block diagram of an example of an analysis pipeline including an ANN for correcting signal data obtained from a nucleic acid sequencing instrument. In the example of FIG. 5, the ANN may operate in the inference mode. The inputs to the artificial neural network, also referred to as the input layer for the neural network, may comprise one or more input channels, each channel including an array of N values. The input channels may include a signal measurement channel and one or more of a simulated signal measurement channel, a signal mask channel, a signal position channel, and four flow order channels. The signal measurement channel may comprise an array of signal measurements result from signal processing applied to the raw data generated during a sequencing run by a nucleic acid sequencing instrument. The signal mask channel may comprise an array having a fixed size with 1's in positions corresponding to an input signal measurement and 0's in positions corresponding to no input signal measurement. The signal position channel may comprise an array of values indicating the positions of the input signal measurements. The position values may be scaled to values between 0.0 and 1.0. For example, for 550 input signal measurements, the signal position values may be 1/550, 2/550, . . . 550/550=1.0. The flow order channels may include one channel for each base, A, C, G and T. A flow order channel for a particular base may be represented an array of 0's and 1's, where a 1 indicates that the particular base in that position in the flow order was flowed to generate the corresponding signal measurement. Table 1 gives an example of four flow order channels for a flow order of A, C, G, C, A and T.

TABLE 1

| FLOW ORDER CHANNEL | A | C | G | C | A | T |
|---|---|---|---|---|---|---|
| A | 1 | 0 | 0 | 0 | 1 | 0 |
| C | 0 | 1 | 0 | 1 | 0 | 0 |
| G | 0 | 0 | 1 | 0 | 0 | 0 |
| T | 0 | 0 | 0 | 0 | 0 | 1 |

The flow order channels include entries corresponding to positions of all the signal measurements in the signal measurement channel, according to the nucleotide that was flowed to obtain each signal measurement. The input channels comprise arrays having the same size and may include padding with zeros. For example, for a channel may have dimensions of 1×576. For example, for a number of input signal measurements of 550, the signal measurement channel would be padded with zeros to a length of 576. The other input channels would also include 550 corresponding values and zero padding to length of 576.

The simulated signal measurement channel may comprise an array of simulated signal measurements predicted using a simulation model that predicts what the expected signal measurements would be for a particular base to be called. The simulation model may be constructed in any suitable fashion to mathematically describe the sequencing process. For example, generating the simulated signal measurements may include one or more features described in U.S. Pat. Appl. Publ. No. 2012/0109598 published May 3, 2012, incorporated by reference herein in its entirety. The simulated signal measurements may be generated from a simulation model that includes model parameters such as a “carry forward” parameter and an “incomplete extension” parameter, as described above.

As shown in FIG. 5, the ANN may be applied to the input channels to produce an output channel comprising an array of signal correction values. Optionally, the ANN may also provide as an output channel corrected signal measurements, for example, in flow space. The signal correction values represent a systematic error component of the input signal measurements. The signal correction values are subtracted from the input signal measurements to remove the systematic error component to produce corrected signal measurements. The corrected signal measurements are input to the base caller. The base caller uses the corrected signal measurements, instead of the original input signal measurements, to determine the base calls for a sequence read. For downstream processing, the sequence reads may be provided to the alignment step and variant calling, as described with respect to FIG. 4.

In some embodiments, the ANN may comprise a convolutional neural network (CNN). For example, the CNN may have a U-Net architecture. See, e.g., Ronneberger et al., U-Net: Convolutional Networks for Biomedical Image Segmentation, arXiv:1505.04597v1′, [cs.CV], 18 May 2015. A U-Net architecture may comprise a plurality of processing layers of an encoding portion, or contracting path, and a decoding portion, or expansive path. The encoding, or contracting, path is a convolutional network that includes multiple convolutional layers, each applying a plurality of convolutions followed by a batch normalizing operation and a non-linear activation function, e.g., Sigmoid Linear Unit (SiLU). For the encoding, or contracting, path one or more of the convolutional layers may be followed by a pooling layer performing max pooling operations. During the contracting, the dimensions of the input signal information is reduced by the max pooling operations while feature information is increased by the convolution operations. The decoding, or expansive, path combines the feature and signal information through a sequence of up-convolutions and concatenations with high-resolution features from the encoding, or contracting, path.

A convolutional layer of a CNN may apply a plurality of convolutions to a given input channel with a plurality of convolutional kernels. In some convolutional layers, the convolutional kernel may have a size of B. A given convolutional kernel is convolved with the array of values of a given input channel to the layer. The convolution operation may include multiply, add and shift operations as follows:
1) Aligning the position of the convolutional kernel of size B with a position in input array of N values input to the layer;
2) Multiplying the weights of the convolutional kernel and the values of the array overlapped by the convolutional kernel to form B products;
3) Adding the B products to give an output value for a corresponding position in an output array for the layer;
4) Shifting the position of the convolutional kernel by a stride value;
5) Repeating steps 2) to 4) to form a convolved array having N values.

The input array may be padded with zeros so that the convolved array size has the same as the input array size. Convolution may include reflecting or reversing the feature map values prior to the convolution calculations. The convolution, as described herein, is consistent with a cross-correlation of the convolutional kernel and the input array. The terms “convolution” and “cross-correlation” are used interchangeably herein.

For multiple input channels, a particular convolutional kernel may be applied to a corresponding one of the input channels. The collection of convolutional kernels applied to multiple input channels is referred to as a filter. Each convolutional kernel “slides” over its respective input channel, producing a per-channel convolved array. The per-channel convolved arrays are then summed together to form a summed array (feature map) for an output channel. Optionally, a bias value may be added to each element of the summed array for an output channel. Multiple output channels for a given layer may be produced. The number of output channels corresponds to the number of filters applied to the input channels of the layer. The weights of the convolutional kernels and bias values may be learned during training mode and fixed at constant values during inference mode.

A batch normalization may be applied to each of the output channels of a given layer. The batch normalization for each output channel is calculated according to Equation (1):

y=(x−E[x]/sqrt(Var[x])*γ+β (1)

Where, y is the normalized value for the element in the output channel, x is the element value in the output channel, E(x) is the expected value, or mean value, of the elements in the output channel, Var(x) is the variance of the elements in the output channel, and γ and β are mean and variance parameters for each output channel that may be learned during training mode. By default, the values of γ may be set to 1 and the values of β may be set to 0.

Batch normalization may be applied before or after an activation function is applied to normalize values for the next layer. In some embodiments, the activation function may be the sigmoid linear unit function (SiLU). The SiLU function is given by equation (2):

SiLU(x)=x/(1+e^{-x}) (2)

where x may be the normalized value or non-normalized value of an output channel. Embodiments of the present disclosure may use other activation functions such as a rectified linear unit function (ReLU), leaky ReLU function, a softmax function, a sigmoid function, and the like.

A pooling layer may apply maximum pooling (MaxPool) operations to the output channel of a convolutional layer that is input to the pooling layer. The MaxPool operation for a kernel size of 2 and stride value of 2 performs the following operations:
1) Align a window with kernel size of 2 with an initial position in the input channel to the pooling layer;
2) Select the maximum of 2 values of the input channel covered by the window for a corresponding position in the output channel of the pooling layer;
3) Shift the alignment of the window by the stride value of 2;
4) Repeat steps 2) and 3) to form the output channel of the pooling layer.
   For the stride value of 2, the size of the output channel of the pooling layer is half the size of the input channel to the pooling layer. Embodiments of the present disclosure may use other pooling operations such as average pooling (AvgPool) operations. For AvgPool operations, the average of the 2 values is calculated in step 2) above, instead of selecting the maximum of 2 values.

Operations by the convolution layers and pooling layers, typically reduce (downs ample) the dimension of the array (feature map) of an input channel or keep it unchanged. To expand the dimensions, another type of CNN layer applies transposed convolution operations to increase (upsample) the dimension of the output array. A 1D transposed convolution operator of kernel size 2 and stride value of 2 applies the following operations to a 1D array of size S of an input channel to the layer:
1) For every element of the input array, the kernel weights are multiplied by the input element values at which the kernel is placed, and the output of the multiplied kernel values are placed at the corresponding locations in the output;
2) After each such multiplication, the outputs are placed over positions in the output array with a gap specified by the stride value;
3) Adding the outputs of the multiplications at corresponding positions to produce the final output array of size 2S.
   The transposed convolution with stride values of 2 doubles the length of the feature map for the output array. For multiple input channels, a transposed convolutional kernel may be applied to a corresponding one of the input channels to form a transposed convolution array per-channel. The per-channel transposed convolution arrays are then summed over the input channels to form an output feature map for the output channel. The weights of the transposed convolution kernels may be learned during a training mode and fixed at constant values during inference mode.

In some embodiments, the CNN may include a Convolutional Block Attention Module (CBAM), between the encoding, or contracting, path and the decoding, or expansive, path. See, e.g., Woo, S. et al. CBAM: Convolutional Block Attention Module, arXiv:1807.06521v2 [cs.CV], 18 Jul. 2018. The U-Net bottleneck part between the contracting path and the expansive path encodes the most powerful and discriminative semantic features. The CBAM includes a spatial-wise attention module and a channel-wise attention module, each applied to an intermediate feature map input to the CBAM. The spatial-wise attention module may comprise a standard convolutional layer with pooling. In the present context, “spatial” corresponds to the temporal dimension of the array of signal measurements. The channel-wise attention module emphasizes inter-channel relationships of features. In more detail, the channel-wise attention module applies max-pooling and average-pooling to the input features to the CBAM on a per channel basis to generate two descriptor arrays, which denote average-pooled features and max-pooled features, respectively. Each descriptor array is input to a multi-layer perceptron (MLP) to produce an average-pooled output feature vector for the average-pooled features and a max-pooled output feature vector for the max-pooled features. The MLP may have two linear layers which may use activation functions. An element-wise summation of the max-pooled output feature vector and the average-pooled output feature vector is followed by sigmoid function to produce a channel attention map, or array of channel attention values. The channel attention map has a length of the number of channels. An element-wise multiplication of the channel attention map and the intermediate feature map input to the CBAM to form a channel-refined feature map. The channel-refined feature map is provided to the spatial-wise attention module. The spatial-wise attention module applies max-pooling and average-pooling to the channel-refined feature map along the channel axis to generate a max-pooled feature map and an average-pooled feature map. The max-pooled feature map and average-pooled feature map are concatenated and convolved by a convolutional layer, followed by a sigmoid function to form a spatial attention map, or array of spatial attention features. The CBAM output is determined by an element-wise multiplication of the spatial attention map and the channel-refined feature map to form the refined output feature map of the CBAM. The weights for the MLP and the convolutional kernels may be learned during the training mode.

The decoding, or expansive, path may combine the feature and signal information by applying up-convolutions and concatenations with high-resolution features from the encoding, or contracting, path. A copy and concatenate step, also called skip connection, may copy the feature map of a channel (layer) at a particular scale in the encoder path and concatenate it with the upsampled feature map of a channel (layer) at the same scale in the decoding path. The concatenated feature maps increase the length of channel.

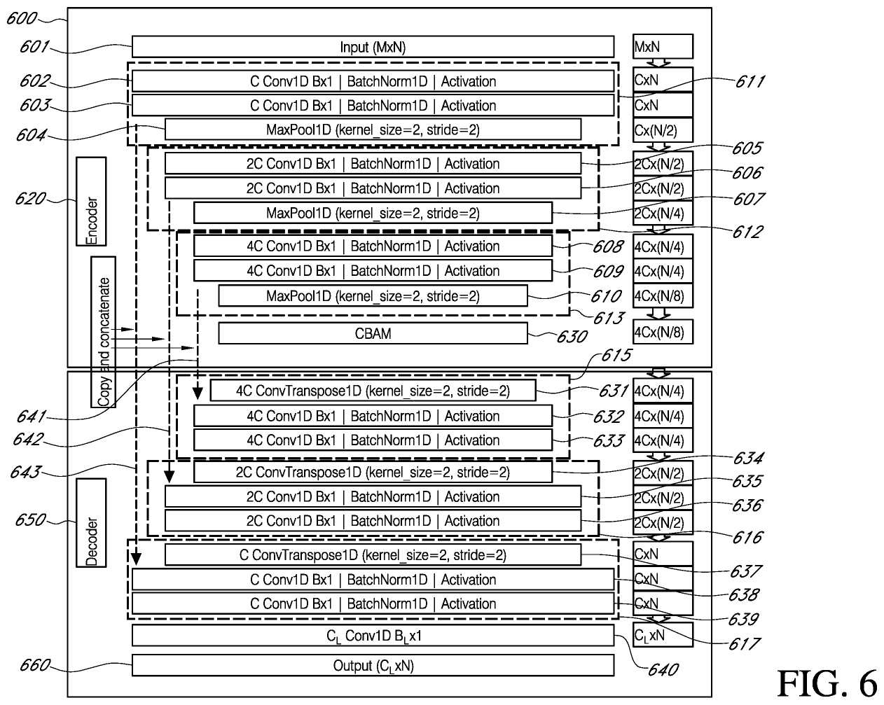

FIG. 6 illustrates an example of a CNN having a U-Net architecture and including a CBAM. The U-Net 600 may include an encoder 620 configured to generate encoded feature maps at multiple scales that are provided to the decoder 650. The decoder 650 combines the feature maps at the different scales to generate the output 660. The copy and concatenate steps, or skip connections, 641, 642, and 643 jump over layers to transfer feature information from the encoder to the decoder. The input layer 601 may receive M input channels, wherein each of the M input channels includes an array of N values, for dimensions of M×N. The encoder 620 may include layer group 611, layer group 612, and layer group 613. The layer groups 611, 612, and 613 of the encoder 620 each include two convolutional layers configured to perform convolution, batch normalization and activation function, such as SiLU, and a pooling layer applying a kernel size of 2 and a stride of 2 for downsampling. The decoder 650 may include layer group 615, layer group 616, and layer group 617. The layer groups 615, 616, and 617 of the decoder 650 each include a convolution transpose layer configured to perform transposed convolution operations of kernel size 2 and stride value of 2 for upsampling and two convolutional layers configured to perform convolution, batch normalization, and activation function, such as SiLU. In some embodiments, the activation function is a SiLU, but embodiments of the present disclosure may use other activation functions such as a leaky ReLU function, a softmax function, a sigmoid function, and the like.

In more detail, at layer 602 of layer group 611, a number C of one-dimensional convolutions are applied to each of the M input channels. Each convolutional kernel has a size of B. A given convolutional kernel is convolved with the values of a given input channel with a stride value of 1. Batch normalization may be applied to each of the C feature maps generated by the convolutions to produce normalized feature maps. An activation function, such as SiLU, is applied to the normalized feature maps to produce the output feature maps for the output channel of layer 602. The scale for layer 602 is C×N. The number of output channels C corresponds to the number of filters applied to the input channels of the layer 602. The output channels of layer 602 are input to layer 603. At layer 603, a number C of one-dimensional convolutions with stride value of 1 are applied to the output channels of layer 602, followed by batch normalization and activation function, such as SiLU, to produce the output feature maps for the C output channels of layer 603. The scale for layer 603 is C×N. The C output channels of layer 603 are input to pooling layer 604. At pooling layer 604, a MaxPool operation having a kernel size of 2 and stride value of 2 is applied to each output channel of layer 603, which reduces the dimension of the feature map in each channel to N/2. The scale for layer 604 is C×(N/2).

The layer group 612 doubles the number of feature channels to 2C and reduces the dimensions of the feature maps to N/4. In more detail, the C output channels of layer 604 are input to layer 605 of layer group 612. A number 2C of one-dimensional convolutions are applied to each of the C output channels from layer 604. Each convolutional kernel has a size of B. A given convolutional kernel is convolved with the values of a given input channel with a stride value of 1. Batch normalization may be applied to each of the 2C feature maps generated by the convolutions to produce normalized feature maps. An activation function, such as SiLU, is applied to the normalized feature maps to produce the output feature maps for the output channel of layer 605. The scale for layer 605 is 2C×(N/2). The number of output channels 2C corresponds to the number of filters applied to the input channels of the layer 605. The output channels of layer 605 are input to layer 606. At layer 606, a number 2C of one-dimensional convolutions with stride value of 1 are applied to the output channels of layer 605, followed by batch normalization and activation function, such as SiLU, to produce the output feature maps for the 2C output channels of layer 606. The scale for layer 606 is 2C×(N/2). The 2C output channels of layer 606 are input to pooling layer 607. At pooling layer 607, a MaxPool operation having a kernel size of 2 and stride value of 2 is applied to each output channel of layer 606, which reduces the dimension of the feature map in each channel to N/4. The scale for layer 607 is 2C×(N/4).

The layer group 613 doubles the number of feature channels from 2C to 4C and reduces the dimensions of the feature maps to N/8. In more detail, the 2C output channels of layer 607 are input to layer 608 of layer group 613. A number 4C of one-dimensional convolutions are applied to each of the 2C output channels from layer 607. Each convolutional kernel has a size of B. A given convolutional kernel is convolved with the values of a given input channel with a stride value of 1. Batch normalization may be applied to each of the 4C feature maps generated by the convolutions to produce normalized feature maps. An activation function, such as SiLU, is applied to the normalized feature maps to produce the output feature maps for the output channel of layer 608. The scale for layer 608 is 4C×(N/4). The number of output channels 4C corresponds to the number of filters applied to the input channels to the layer 608. The output channels of layer 608 are input to layer 609. At layer 609, a number 4C of one-dimensional convolutions with stride value of 1 are applied to the output channels of layer 608, followed by batch normalization and activation function, such as SiLU, to produce the output feature maps for the 4C output channels of layer 609. The scale for layer 609 is 4C×(N/4). The 4C output channels of layer 609 are input to pooling layer 610. At pooling layer 610, a MaxPool operation having a kernel size of 2 and stride value of 2 is applied to each output channel of layer 609, which reduces the dimension of the feature map in each channel to N/4. The scale for layer 610 is 4C×(N/8).

The 4C output channels of pooling layer 610 provide the intermediate feature map input to the CBAM 630. As described above, the CBAM applies a channel-wise attention module and a spatial-wise attention module to the output from pooling layer 610. The channel-wise attention module uses a parameter which defines the size of the output of the first linear layer which corresponds to the size of the input for the second linear layer of the MLP. The channel-wise attention module produces a channel attention map with dimension of 4C. The spatial-wise attention module applies kernel size 7 and stride value of 1 with 2 input channels, one from max-pooling and one from average-pooling, to the channel-refined feature map, and one output channel in its convolutional layer. The spatial-wise attention module produces a spatial attention map with dimension of N/8. The CBAM generates the refined output feature map having a scale of 4C×(N/8).

In more detail for the decoder 650, the 4C channels of the refined output feature map of the CBAM are input to the convolution transpose layer 631 of layer group 615. The convolution transpose layer 631 is configured to perform transposed convolution operations of kernel size 2 and stride value of 2 for upsampling the features of the 4C input channels by a factor of 2. The scale for layer 631 is 4C×(N/4). The encoded features of layer 609, having the same scale of 4C×(N/4), are concatenated 641 with the upsampled features of layer 631 to form concatenated features. The concatenated features are provided to the convolutional layer 632. At layer 632, a number 4C of one-dimensional convolutions are applied to the concatenated features for each of the 4C channels. Each convolutional kernel has a size of B. A given convolutional kernel is convolved with the values of a given input channel with a stride value of 1. Batch normalization may be applied to each of the 4C feature maps generated by the convolutions to produce normalized feature maps. An activation function, such as SiLU, is applied to the normalized feature maps to produce the output feature maps for the output channels of layer 632. The scale for layer 632 is 4C×(N/4). The number of output channels 4C corresponds to the number of filters applied to the input channels to the layer 632. The output channels of layer 632 are input to layer 633. At layer 633, a number 4C of one-dimensional convolutions with stride value of 1 are applied to the output channels of layer 632, followed by batch normalization and activation function, such as SiLU, to produce the output feature maps for the 4C output channels of layer 633. The scale for layer 633 is 4C×(N/4). The 4C output channels of layer 633 are input to layer 634.

The layer group 616 halves the number of feature channels to 2C and increases the dimensions of the feature maps to N/2. In more detail, the 4C output channels of layer 633 are input to layer 634 of layer group 616. The convolution transpose layer 634 is configured to perform transposed convolution operations of kernel size 2 and stride value of 2 for upsampling the feature maps of the 4C input channels by a factor of 2 and produce 2C output channels. The scale for layer 634 is 2C×(N/2). The encoded features of layer 606, having the same scale of 2C×(N/2), are concatenated 642 with the upsampled features of layer 634 to form concatenated features. The concatenated features are provided to the convolutional layer 635. At layer 635, a number 2C of one-dimensional convolutions are applied to the concatenated features for each of the 2C channels. Each convolutional kernel has a size of B. A given convolutional kernel is convolved with the values of a given input channel with a stride value of 1. Batch normalization may be applied to each of the 2C feature maps generated by the convolutions to produce normalized feature maps. An activation function, such as SiLU, is applied to the normalized feature maps to produce the output feature maps for the output channel of layer 635. The scale for layer 635 is 2C×(N/2). The number of output channels 2C corresponds to the number of filters applied to the input channels to the layer 635. The output channels of layer 635 are input to layer 636. At layer 636, a number 2C of one-dimensional convolutions with stride value of 1 are applied to the output channels of layer 635, followed by batch normalization and activation function, such as SiLU, to produce the output feature maps for the 2C output channels of layer 636. The scale for layer 636 is 2C×(N/4). The 2C output channels of layer 636 are input to layer 637.

The layer group 617 halves the number of feature channels to C and increases the dimensions of the feature maps to N. In more detail, the 2C output channels of layer 636 are input to layer 637 of layer group 617. The convolution transpose layer 637 is configured to perform transposed convolution operations of kernel size 2 and stride value of 2 for upsampling the feature maps of the 2C input channels by a factor of 2 and produce C output channels. The scale for layer 637 is C×N. The encoded features of layer 603, having the same scale of C×N, are concatenated 643 with the upsampled features of layer 637 to form concatenated features. The concatenated features are provided to the convolutional layer 638. At layer 638, a number C of one-dimensional convolutions are applied to the concatenated features for each of the C channels. Each convolutional kernel has a size of B. A given convolutional kernel is convolved with the values of a given input channel with a stride value of 1. Batch normalization may be applied to each of the C feature maps generated by the convolutions to produce normalized feature maps. An activation function, such as SiLU, is applied to the normalized feature maps to produce the output feature maps for the output channel of layer 638. The scale for layer 638 is C×N. The number of output channels C corresponds to the number of filters applied to the input channels to the layer 638. The output channels of layer 638 are input to layer 639. At layer 639, a number C of one-dimensional convolutions with stride value of 1 are applied to the output channels of layer 638, followed by batch normalization and activation function, such as SiLU, to produce the output feature maps for the C output channels of layer 639. The scale for layer 639 is C×N. The C output channels of layer 639 are input to layer 640. At the last layer 640, each convolutional kernel has a size of B_{L}. At layer 640, a number C_{L} of B_{L}×1 convolutional kernels are applied to each of the C input channels to produce a C_{L}×N output 660.

The last layer 640 may use a convolutional layer instead of a linear layer to do regression for the output channels. For example, the convolutional kernel may have a size of B_{L}=1 to reduce dimensionality and the output channel may have a size C_{L}, e.g. to C_{L}=3, to output C_{L} channels. During inference mode, one of the C_{L} output channels provides the signal correction values. During training mode, all of the C_{L} output channels, e.g. C_{L}=3, may be used for multi-task learning to improve overall model performance. For example, the C_{L}=3 output channels of the last layer 640 used during training mode may include an array of signal correction values, an array of labeled simulated signal measurements and an array of maximum allowed residual values. The simulation model may be applied to a labeled base sequence to generate the labeled simulated signal measurements, as described below. The maximum allowed residual values are the maximum error allowed for calling homopolymer lengths corresponding to labeled measurements, as described below.

During inference mode, the output 660 provides an array of signal correction values having dimension of 1×N. As described above with respect to FIG. 5, the signal corrections values are subtracted from the signal measurements output from the signal processing module. The signal correction values are subtracted from the input signal measurements to remove the systematic error component to produce corrected signal measurements. The corrected signal measurements are input to the base caller.

Examples of parameters for the U-Net of FIG. 6 are given in Table 2.

TABLE 2

| PARAMETER | SYMBOL | VALUE | RANGE |
|---|---|---|---|
| Number of input channels | M | 8 | |
| Number of elements in | N | 576 | |
| input channel | | | |
| Size of a convolutional | B | 7 | 3 to 9 |
| kernel | | | |
| Size of last layer's | B_{L} | 1 | 1 to 9 |
| convolutional kernel | | | |
| Minimum number of | C | 48 | 48 to 128 |
| output channels for a layer | | | |
| Number of output channels | C_{L} | 3 | 1 to 3 |
| for last layer | | | |

In an example, Table 3 lists parameters for the U-Net of FIG. 6.

TABLE 3

| Line No. | Layer (type) | Output Shape | Param # |
|---|---|---|---|
| 1. | Conv1d-1 | [−1, 48, 576] | 2,688 |
| 2. | BatchNorm1d-2 | [−1, 48, 576] | 96 |
| 3. | SiLU-3 | [−1, 48, 576] | 0 |
| 4. | Conv1d-4 | [−1, 48, 576] | 16,128 |
| 5. | BatchNorm1d-5 | [−1, 48, 576] | 96 |
| 6. | SiLU-6 | [−1, 48, 576] | 0 |
| 7. | MaxPool1d-7 | [−1, 48, 288] | 0 |
| 8. | Conv1d-8 | [−1, 96, 288] | 32,256 |
| 9. | BatchNorm1d-9 | [−1, 96, 288] | 192 |
| 10. | SiLU-10 | [−1, 96, 288] | 0 |
| 11. | Conv1d-11 | [−1, 96, 288] | 64,512 |
| 12. | BatchNorm1d-12 | [−1, 96, 288] | 192 |
| 13. | SiLU-13 | [−1, 96, 288] | 0 |
| 14. | MaxPool1d-14 | [−1, 96, 144] | 0 |
| 15. | Conv1d-15 | [−1, 192, 144] | 129,024 |
| 16. | BatchNorm1d-16 | [−1, 192, 144] | 384 |
| 17. | SiLU-17 | [−1, 192, 144] | 0 |
| 18. | Conv1d-18 | [−1, 192, 144] | 258,048 |
| 19. | BatchNorm1d-19 | [−1, 192, 144] | 384 |
| 20. | SiLU-20 | [−1, 192, 144] | 0 |
| 21. | MaxPool1d-21 | [−1, 192, 72] | 0 |
| 22. | Flatten-22 | [−1, 192] | 0 |
| 23. | Linear-23 | [−1, 12] | 2,316 |
| 24. | ReLU-24 | [−1, 12] | 0 |
| 25. | Linear-25 | [−1, 192] | 2,496 |
| 26. | Flatten-26 | [−1, 192] | 0 |
| 27. | Linear-27 | [−1, 12] | 2,316 |
| 28. | ReLU-28 | [−1, 12] | 0 |
| 29. | Linear-29 | [−1, 192] | 2,496 |
| 30. | ChannelGate-30 | [−1, 192, 72] | 0 |
| 31. | ChannelPool-31 | [−1, 2, 72] | 0 |
| 32. | Conv1d-32 | [−1, 1, 72] | 14 |
| 33. | BatchNorm1d-33 | [−1, 1, 72] | 2 |
| 34. | BasicConv-34 | [−1, 1, 72] | 0 |
| 35. | SpatialGate-35 | [−1, 192, 72] | 0 |
| 36. | CBAM-36 | [−1, 192, 72] | 0 |
| 37. | ConvTranspose1d-37 | [−1, 192, 144] | 73,920 |
| 38. | Conv1d-38 | [−1, 192, 144] | 516,096 |
| 39. | BatchNorm1d-39 | [−1, 192, 144] | 384 |
| 40. | SiLU-40 | [−1, 192, 144] | 0 |
| 41. | Conv1d-41 | [−1, 192, 144] | 258,048 |
| 42. | BatchNorm1d-42 | [−1, 192, 144] | 384 |
| 43. | SiLU-43 | [−1, 192, 144] | 0 |
| 44. | ConvTranspose1d-44 | [−1, 96, 288] | 36,960 |
| 45. | Conv1d-45 | [−1, 96, 288] | 129,024 |
| 46. | BatchNorm1d-46 | [−1, 96, 288] | 192 |
| 47. | SiLU-47 | [−1, 96, 288] | 0 |
| 48. | Conv1d-48 | [−1, 96, 288] | 64,512 |
| 49. | BatchNorm1d-49 | [−1, 96, 288] | 192 |
| 50. | SiLU-50 | [−1, 96, 288] | 0 |
| 51. | ConvTranspose1d-51 | [−1, 48, 576] | 9,264 |
| 52. | Conv1d-52 | [−1, 48, 576] | 32,256 |
| 53. | BatchNorm1d-53 | [−1, 48, 576] | 96 |
| 54. | SiLU-54 | [−1, 48, 576] | 0 |
| 55. | Conv1d-55 | [−1, 48, 576] | 16,128 |
| 56. | BatchNorm1d-56 | [−1, 48, 576] | 96 |
| 57. | SiLU-57 | [−1, 48, 576] | 0 |
| 58. | Conv1d-58 | [−1, 3, 576] | 147 |

Relating the entries in Table 3 to FIG. 6, line nos. 1, 2 and 3 give the parameters for layer 602; line nos. 4, 5 and 6 give the parameters for layer 603; line no. 7 gives the parameters for layer 604; line nos. 8, 9 and 10 give the parameters for layer 605; line nos. 11,12 and 13 give the parameters for layer 606; line 14 gives the parameters for layer 607; line nos. 15, 16 and 17 give the parameters for layer 608; line nos. 18, 19 and 20 give the parameters for layer 609; line 21 gives the parameters for layer 610; line nos. 22-36 give the parameters for the CBAM layer 630; line no. 37 gives the parameters for layer 631; line nos. 38, 39 and 40 give the parameters for layer 632; line nos. 41, 42 and 43 give the parameters for layer 633; line no. 44 gives the parameters for layer 634; line nos. 45, 46 and 47 give the parameters for layer 635; line nos. 48, 49 and 50 give the parameters for layer 636; line no. 51 gives the parameters for layer 637; line nos. 52, 53 and 54 give the parameters for layer 638; line nos. 55, 56 and 57 give the parameters for layer 639; and line no. 58 gives the parameters for layer 640. In this example, the total number of U-Net parameters is 1,651,339, all of which are trainable.

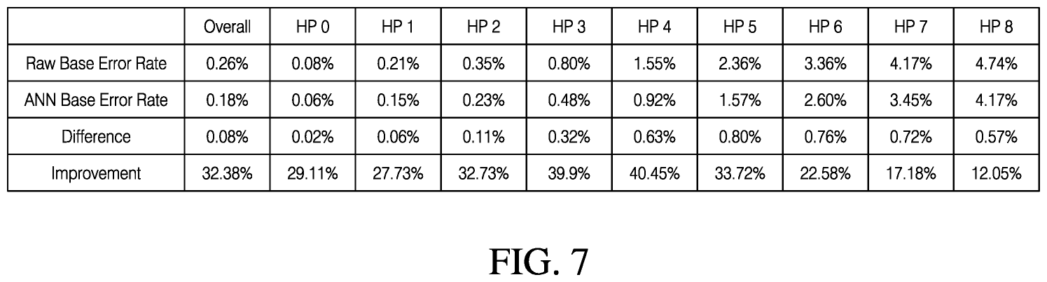

FIG. 7 shows and example of improvements in base error rates achieved using the ANN in the analysis workflow, e.g. the workflow of FIG. 5, versus no ANN in the analysis workflow, e.g. the workflow of FIG. 4. The Raw Base Error Rate gives the percentage of base calls that are errors when the workflow did not include an ANN, e.g. the workflow of FIG. 4. The ANN Base Error Rate gives the percentage of base calls that are errors when an ANN was included in the workflow, e.g. the workflow of FIG. 5. The Difference was calculated between the Raw Base Error Rate and the ANN Base Error Rate. The Improvement is the ratio of the Difference to the Raw Base Error Rate. These results show an overall reduction in base error rate of 32.8%, which shows the improvement in base calling accuracy when the ANN is used for signal correction. Furthermore, the base error rates at each homopolymer length showed improvements in base calling accuracy for homopolymer lengths from 1 to 8. The improvement in accuracy is also shown when there was no incorporation of a nucleotide (HP=0).

As noted above, artificial neural networks for performing signal correction operations using neural network architectures in accordance with embodiments of the present disclosure are trained using training data sets. The training data sets match sequencing data obtained from sequencing experiments applied to a sample of a known reference that has well-characterized genotyping information, to corresponding known truth base sequences. Industry standard cell line samples for NGS benchmarking, such as NA12878 (a.k.a HG001) and NA24385 (a.k.a. HG002), may be used as references for training the ANN. For example, labelling the sequence data of NA12878 library provides the corresponding “ground truth”. Other well characterized samples, either publicly available or proprietary, can be used for training. For example, a sample may be derived from an assay such as the AMPLISEQ™ CARRIERSEQ™ ECS panel (Thermo Fisher Scientific) for expanded carrier screening (ECS) or the AMPLISEQ™ Exome panel (Thermo Fisher Scientific) for genome and DNA sequencing.

Training an ANN generally involves initializing the ANN (e.g., setting the weights in the network, such as the weights in the convolutional kernels, to random values), and providing the training input data to the network. The output of the network is then compared against the labeled training data to generate an error signal (e.g., a difference between the current output and the “ground truth” output), and a backpropagation algorithm is used with gradient descent to update the weights, over many iterations, such that the network computes a result closer to the desired ground truth.

FIG. 8 illustrates an example process and system for labeling and training. In an example, the system can act as a labeling system to label measurements for use in training a neural network. In another example, the process and system can act as a training system and can be utilized to train a neural network, such as a U-Net convolutional neural network, to predict or provide adjustments or signal correction values. Under normal operation, a sequencer can generate signals associated with the incorporation of nucleotides complementary to a target sequence, referred to herein as flow measurements. Either using the sequencer or a separate computational system, the signal from the sequencer can be converted into a sequence of bases, referred to herein as base calling. Often, the sequencer sequences more than one copy of a sequence or sequences the sequence more than one time.

FIG. 8 illustrates an example process and system for labeling and training. In an example, the system can act as a labeling system to label measurements for use in training a neural network. In another example, the process and system can act as a training system and can be utilized to train a neural network, such as a U-Net convolutional neural network, to predict or provide adjustments or signal correction values. Under normal operation, a sequencer can generate signals associated with the incorporation of nucleotides complementary to a target sequence, referred to herein as flow measurements. Either using the sequencer or a separate computational system, the signal from the sequencer can be converted into a sequence of bases, referred to herein as base calling. Often, the sequencer sequences more than one copy of a sequence or sequences the sequence more than one time.

In addition, the process and system can align the reads or sequences with each other and optionally with an expected sequence, referred to herein as alignment. The labeling system can utilize either the aligned read sequences from the alignment process or can use the raw base sequence from the base calling process to associate the read sequences and the associated signal from the sequencer with a known sequence, its variants, and a simulated signal measurement, such as a predicted signal measurement in flow space or a predicted flow measurement. The labeling system can further determine a signal correction value to associate with the flow measurement and associated read sequence.

Such a process and system find particular use when the same region of a genome, for example, is amplified and sequenced many times and stored in many reads. For example, targeted sequencing using library preparation methods, such as Ampliseg™ by Ion Torrent, provides many copies of targeted regions that are sequenced, providing many stored reads of the same targeted region. Alternatively, random fragments of the genome can be used when enough copies of the same fragment (i.e., group) are obtained.

Training a neural network, such as a U-Net convolutional neural network, utilizes a significant amount of data. A labeling system can be utilized to retrieve measurements and label the measurements with the appropriate expected values and underlying parameters. Such labeling can be complicated by variability in the expected values of the measurements.

In an example, a measured series or sequence can be compared to a set of series or sequences to determine which series or sequence most closely matches the measured series or sequence. Some series or sequences within the set of series or sequences may include one or more positions that have more than one valid or expected value, referred to as variants. Each of these variants represents the valid or expected value. As such, the variants represent valid results to be detected and do not represent error. The measured series or sequence can be matched to the variant within the matched series or sequence. Proper labeling of the measured series or sequence with the appropriate sequence and variant allows for the detection of such variants instead of treating the variants as errors to be corrected. For example, a series can be a series of signal measurements in flow space. In another example, a sequence can be a sequence of base calls.

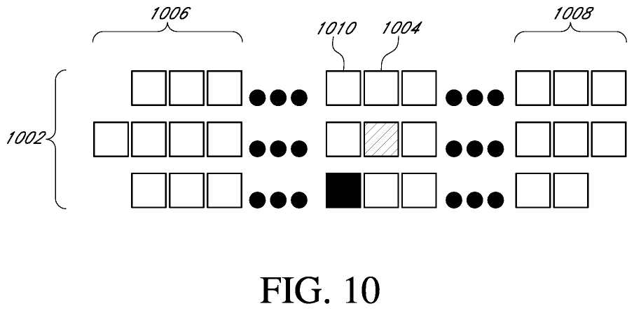

For example, FIG. 10 illustrates a set of measured series or sequences 1002. At a position 1004, the measurements 1002 can have more than one acceptable value. Each value of the more than one values represents expected variants within the sample. On the other hand, the difference in value, for example, at position 1010, can represent an error in the measurement. It is desired that a neural network be trained that recognizes or corrects for errors, such as at position 1010, and that does not alter the measurements at position 1004, which represents permissible variants within the measurement. In addition, the measured sequences may have terminal ends 1006 or 1008. The terminal ends 1006 or 1108 may have same number of measured positions. Alternatively, the measured sequences may have similarities within the middle of the sequence and a different number of positions at the ends 1006 or 1008, for example, due to measurement errors. Often the differences occur in the region associated with amplicon primers. As illustrated, the measurements, such as nucleic acid sequences, are aligned with other closely matching measurements or sequences. As described below, such sequences can be matched with known or expected sequences and their associated variants.

Such labeling techniques find particular use when sequencing biological systems, such as nucleic acids or proteins. For example, segments of a larger nucleic acid sample can be amplified to provide multiple copies of these nucleic acid segments. Such segments can then be sequenced to determine which alleles are present within the sample. The alleles relate to which variants are present on different sequences derived from the segments. In a particular example, the amplification is designed to amplify specific amplicon sequences found at regions of the sample nucleic acid. The measured sequences can be matched with expected amplicon sequences of the set of amplicon sequences. Once the most likely amplicon sequence is matched with the measured sequence, variants within the amplicon sequence are compared to the measured sequence to determine which variants represent an expected or true value to associate with the measured sequence. As such, the measured sequence can be labeled with the matched amplicon sequence and the associated variant.

As described above, it is desired to correct errors in a flow measurement (signal measurements in flow space) prior to determining a sequence based on the flow measurement. The flow measurement is a measurement indicative of incorporation events in response to the flow of nucleotides in an ordered series in flow space. Flow space represents the coordinate system including a series of nucleotide flows based on the ordered flow of nucleotides through the system that may cause incorporations. How measurements can be utilized to determine a base sequence that can be matched with a sequence of a set of sequences in base space and further matched with variants within the matched sequence. Base space represents the coordinate system including a position of bases within a sequence. The matched sequence and associated variants can then be used to generate an expected flow measurement. The expected flow measurement, flow measurement, and flow order can be utilized to train a neural network to provide for correction in the flow measurements signal to improve the measurement of sequences based.

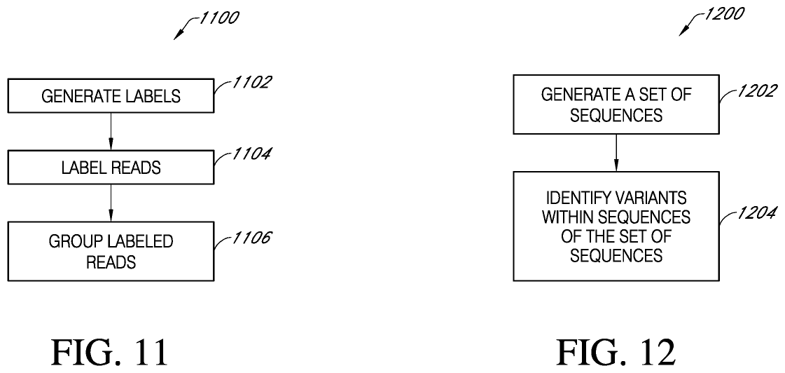

To label measurements, a set of expected or acceptable values is generated and then associated with the measurements that closely match the expected values. The label measurements can then be grouped and statistical values determined about the labeled measurements. For example, as illustrated in FIG. 11, a method 1100 includes generating labels, as illustrated at block 1102. Each measurement or read sequence can be labeled with a select label from the set of generated labels, as illustrated at block 1104. For example, a sequence associated with the read or measurement can be matched to a sequence of a set of sequences. The matched sequence, associated variants, and generated or predicted flow measurements can be used to label a read sequence and its associated flow measurement array. Optionally, a series of residual values can be determined based on the difference between the flow measurement and the predicted flow measurement. As illustrated at block 1106, the labeled reads can be grouped, and statistics can be determined about labeled reads within a group. For example, average residual values at each position within the series of residual values can be determined, as well as variance of the flow measurement or residual values at that position or over all positions.

In particular, a set of labels can be generated, as illustrated at block 1102 of FIG. 11. In an example, FIG. 12 illustrates a method 1200 to generate labels to associate with reads. As illustrated at block 1202, a set of sequences can be generated. For example, a set of nucleic acid sequences can be generated or a set of protein sequences can be generated.

In a particular example, a set of amplicon sequences can be generated based on expected sequences or segments derived from a nucleic acid sample. For example, each of the amplicon sequences can represent the expected sequence of a segment or region of a chromosome. In an example, such chromosomes may be well characterized in a public database or may be proprietary.

Some sequences of the set of sequences can include variants at positions within the sequence. As illustrated at bock 1204, the variants within the sequences can be identified. For example, there may be variants at known positions on a chromosome. Amplicon sequences incorporating those known positions can be identified and the variants within that amplicon identified. The variants can represent single nucleotide polymorphisms (SNP), multi-nucleotide polymorphism (MNP), insertions or deletions (INDELs), among other variants.

For example, in chromosome 1 (chr1) of the human genome, depending on the cell line (e.g., HG001 or HG002), there are known single nucleotide polymorphisms (SNP) at chr1:11794698 and at chr1:11794839. The reference genome can be stored in a reference genome file (.fasta or .fasta.fai). For well characterized genomes, such variants are known and can be identified. Amplicons can be designed that overlap with known variants. Such amplicons can be defined in a BED file (.bed). Each amplicon defined in the BED file can be compared (walked) to determine which positions within the amplicon include a variant and what the permissible values of that variant are. In an example, the true variants and associated amplicons can be defined in a true-variant variant call format (VCF) file (.vcf)

In the example, there are two possible ground truth base sequences (on the forward strand) in the amplicon, represented as:

{phased_seq_1}
(SEQ ID NO: 1)
{″TTCTTCTGCCCTCCCGCTCCCAAGAACAAAGAT″ + ″A″ +
″TATTTGCAAGGAAGGTCTGCAGGCCCTCACCAGCGGCCGTTAGGGAACTCGTCCCACTCCT
GGGTACGGTAGATGTAACTCTTTGGTCTGGAGGCCCAGAAGATGGGACGTACATCTTCCTCT
CGGCGCTTGGGGTGGGC″ + ″G″ +
″CTGAGAGCCCAGGGTAGGGGACGCCTGGGTGAGGATGGGGACAGAGAATTG″}
{phased_seq_2}
(SEQ ID NO: 2)
{″TTCTTCTGCCCTCCCGCTCCCAAGAACAAAGAT″ + ″G″ +
″TATTTGCAAGGAAGGTCTGCAGGCCCTCACCAGCGGCCGTTAGGGAACTCGTCCCACTCCT
GGGTACGGTAGATGTAACTCTTTGGTCTGGAGGCCCAGAAGATGGGACGTACATCTTCCTCT
CGGCGCTTGGGGTGGGC″ + ″A″ +
″CTGAGAGCCCAGGGTAGGGGACGCCTGGGTGAGGATGGGGACAGAGAATTG″}

where {phased_seq_1} and {phased_seq_2} represent the truth of amplicon insert sequences (expected insert sequences) generated by the first and second phased alleles, respectively.

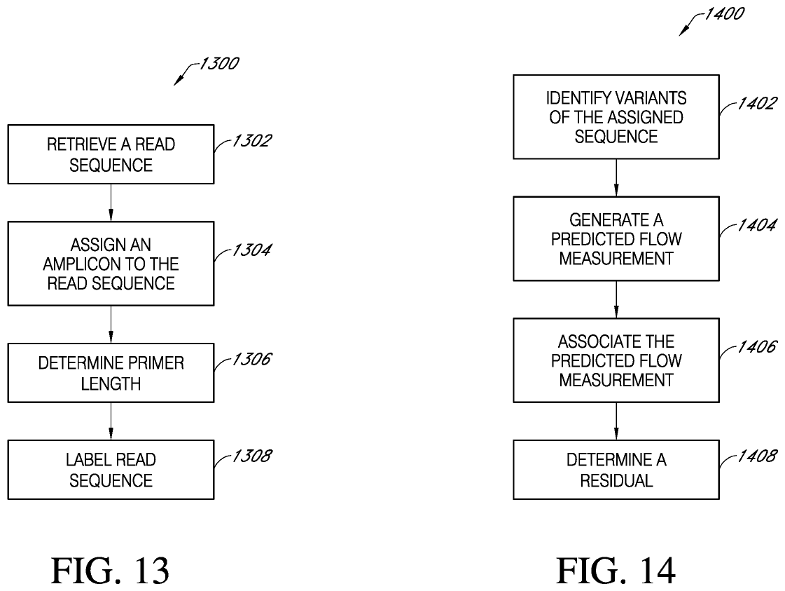

Returning to FIG. 11, the reads may be labeled, as illustrated at block 1104. For example, read sequences can be matched with expected read sequences and the variants within the read sequence can be identified. In an example illustrated in FIG. 13, a method 1300 includes retrieving a read sequence, as illustrated at 1302. For example, the read sequence can be derived from a flow measurement (signal measurements in flow space). In a particular example, the flow measurement can be measured by flowing an ordered sequence of nucleotides (flow order) and measuring incorporation of such nucleotides. The flow measurement can then be converted into a nucleic acid sequence.

In some embodiments, targeted regions of a genome can be sequenced. In an example, amplicons can be used to amplify target regions of sample nucleic acids. For example, a known set of amplicons can be used to amplify target regions of a nucleic acid sample, and each of the read sequences resulting from sequencing the amplified target regions include at least a portion of the amplicon used to target that region. As illustrated at block 1304, an amplicon can be assigned to the read sequence. For example, an amplicon/primer of a set of expected amplicons can be matched with the read sequence. In an example, an amplicon matching algorithm can be found in the Torrent Variant Caller available from ION Torrent of Thermo Fisher Scientific, Inc.

At least a portion of the gene specific or amplicon primer can remain on the nucleic acid sequenced to form the read sequence. As illustrated at block 1306, the primer lengths can be determined for a given read sequence. To generate an expected flow space flow measurement based on the identified amplicon, the primers on the 5′ end and the 3′ end of the read are determined. In some examples, the primer region does not contain variants. As a result, the primer sequences can be determined by the reference genome and the length of the primers. Ideally, the lengths of the primers can be inferred by the amplicon start/last positions and the read alignment start/end positions. However, sequencing errors, soft clipping, or other alignment artifacts can affect the start/end positions of the read. In general, the 3′ end read can be noisier and suffer from homopolymer insertions and deletions (HP-INDEL) and soft clipping and quality trimming Optionally, the length of the 3′ primer of a read can be estimated by the 5′ primer of reads on opposite strands. For example, segments of both strands of a double helix DNA can be sequenced, and the 3′ prime ends inferred from the 5′ end of the opposite strand.

As illustrated at block 1308, the read sequence can be labeled. In an example, the read sequence and associate flow measurement can first be labeled in base space, identifying the amplicon and variants within the amplicon associated with the read sequence. In addition, the read sequence can be further labeled with an expected flow measurement generated based on the matched amplicon sequence (predicted flow measurement) and variants, as well as model parameters and nucleotide flow order. Further, the read sequence can be labeled with an indicator of strand direction, the 5′ end overhead, the 3′ end overhead, or a residual array.

For example, as illustrated in FIG. 14, a method 1400 can include identifying the variant of an assigned sequence, as illustrated at block 1402. For example, an assigned amplicon can have positions that include variants. The read sequence is matched with the most likely amplicon and assigned variants, and the assigned amplicon and variants are used to label the read sequence in base space.

In some cases, errors in the base calling make identification of the amplicon and associated variants difficult. As such, a predicted flow measurement (signal measurements in flow space) can be generated based on flow order and the matched amplicon, as illustrated at block 1404. For example, a simulation model and associated model parameters, such as a “carry forward” parameter and an “incomplete extension” parameter, can be used to generate a predicted flow measurement.

Based on the most likely match of the predicted flow measurement, the amplicon and associated variants can be associated with the read sequence and its associated flow measurement, as illustrated at block 1406. For example, the read sequence and flow measurement can be labeled with the predicted flow measurement, and optionally with amplicon, variant, nucleotide flow order, strand direction, or modeling parameters. For example, a predicted flow measurement can be generated from the matched amplicon, and most likely variant using models. In an example, the model and associated parameters can be used along with the nucleotide flow order to provide a predicted flow measurement.

As illustrated at block 1408, the residual or difference between the flow measurement and the predicted flow measurement can be determined for each position within the series of the flow measurement and predicted flow measurement. A hash key can be generated for each read sequence according to the read labeling results.

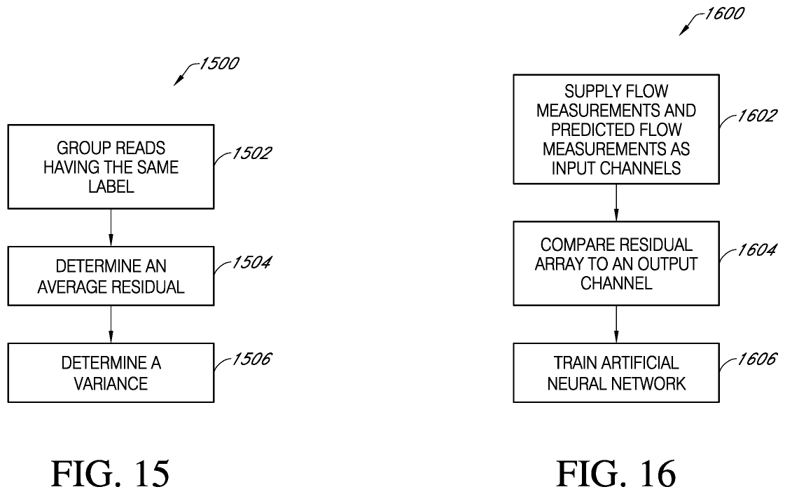

Returning to FIG. 11, the labeled reads can be grouped, as illustrated at block 1106. It is assumed that reads that support the same base sequence and have the same flow space pattern share statistical characteristics of noise. As such, the reads can be grouped using a hash key that was generated in the read labeling. As illustrated in FIG. 15, a method 1500 includes grouping reads having the same label, as illustrated at block 1502. In particular, reads with the same strand direction, assigned amplicon or gene-specific primers, and sequence can form a group. The group can be labeled with total read counts, mead residual array, variance array, mean predicted flow measurement, variance in the predicted flow measurement, or model parameters.

As illustrated at block 1504, an average residual at each position in the series of the flow measurement and the predicted flow measurement can be determined. In particular, a mean of the residual at each position can be determined. In an example, the average residual can be provided as an array.

As illustrated at block 1506, a variance at each position can also be determined. The variance at each flow can indicate how noisy the flow measurements are at that flow (i.e., each flow of a nucleotide). Residuals and measurements depend on the phasing model. Different reads may have different model parameters that may or may not further induce variability in the residuals. Variability of model parameters can cause minor variability in the predicted signals. The variance at each flow can be used to analyze or regularize the regression loss during the training steps. In an example, the variance can be provided as an array.

As such, a set of read sequences determined based on flow measurements are labeled with an associated expected flow measurement and, optionally, an amplicon, variant, nucleotide flow order, residual or mean residual array, or model parameters, such as phase modeling parameters. Such labeled read sequences can be utilized to train a neural network to predict adjustment factors (e.g., signal correction values) that can be used to adjust flow measurements that are used to provide improved base calling. Optionally, the ANN can provide a corrected flow measurement.

For example, FIG. 16 illustrates a method 1600 to train a neural network, such as a U-Net convolutional neural network, to determine an adjustment factor (e.g., signal correction values). As illustrated at block 1602, a training system can supply as input channels a flow measurement as an array and a predicted flow measurement as an array. Input channels can further include nucleotide flow order, position within the flow order, a series mask, a matched sequence, a measured sequence, a variance array, or other parameters.

In an example, a channel is represented by an array. For example, flow measurement can be represented by 1×N array in which N is a number, such as an even number greater than the number of flows within a flow measurement Similarly, the predicted flow measurement can be represented by a 1×N array. In another example, the flow order can be represented by 4×N array (e.g., flow order channels) in which the flow of each nucleotide is recorded in each of the rows of the channel. In a particular example, a position within the flow order (e.g., signal position channel) and a series mask (e.g., signal mask channel) are provided as input channels. For example, a signal position channel can be dedicated to indicate position having a range of values from 0 to 1. Owing to various errors or conditions of the simulation model, the expected signal generated by the simulation model can vary based on position. In addition, the size of the array may be greater than the number of data points, for example, an even number greater than the number of data points. A series mask (e.g., signal mask channel) can be used to indicate which columns of the array contain data and which are set to null.

As illustrated at block 1604, an array of generated residual values (signal correction values) can be provided as an output channel. During training, the generated flow measurement of a flow measurement (signal values) can also be provided as an output channel. It has been found that utilizing both the generated residual array and another factor, such as the generated flow measurement, as outputs to be predicted by the neural network improves the training of the neural network. When the neural network is used for inference, output associated with the other factor can be disregarded. In an example, the output signal correction values can be compared to the residual array or the mead residual array. For example, a difference can be determined. In another example, the generated flow measurement can be compared to the predicted flow measurement, for example, a difference can be determined.

As illustrated at block 1606, a convolutional neural network can be iteratively trained using the input channels and the output channels. During inference mode, the prediction of the second factor, such as the predicted flow measurements or variance array, can be ignored, and the predicted adjustment factors or predicted residuals can be used to adjust the flow measurements to improve base calling. In an example, the training optimizes the parameters of the ANN to minimize the mean squared error. For example, the training minimizes the mean square error between the residual array or mean residual array and the output (signal correction values) from the neural network. In another example, the training also minimizes the mean square error between the predicted flow measurements and the output (signal values) from the neural network. Optionally, other output channels, such as a variance channel, can be used to improve training The methods for training may comprise a machine learning algorithm, for example, a stochastic gradient descent (SGD) algorithm, Adam, Adadelta, Adagrad or other adaptive learning algorithm.

In an example, the training set can include between 1,000 and 10,000,000 labeled reads. For example, the training set can include 10,000 to 5,000,000 labeled reads, such as 100,000 to 5,000,000 labeled reads or 500,000 to 5,000,000 labeled reads. The labeled reads can be grouped into 1,000 to 100,000 groups. For example, the labeled reads can be grouped into 1,000 to 50,000 groups, such as 5,000 to 50,000 groups. A majority of the groups have at least 10 reads, such as at least 20 reads or at least 30 reads, but generally, not greater than 10,000 reads. In an example, the mean group size is in a range of 10 to 100 reads, such as a range of 20 to 100 reads or a range of 30 to 80 reads. In an example, labeled reads of the training set can have between 50 and 1000 bases in base space, such as 50 to 600 bases or 100 to 400 bases. In flow space, the flow measurements associated with reads can have between 100 and 10,000 flows, such as between 100 and 5,000 flows or between 400 and 2,000 flows.

Returning to FIG. 8, for training an artificial neural network, the simulated signal measurements, such as the predicted flow measurements (e.g., simulated signal measurement channel), a signal mask (e.g., signal mask channel), a signal position (e.g., signal position channel), a nucleotide flow order (e.g., flow order channels), and a measured signal (e.g., signal measurement channel), such as the flow measurements are provided as inputs, such as input channels, to the artificial neural network. The output from the artificial neural network is compared with the signal correction values, such as the mean residual array, providing a training loss which can be used to update parameters of the artificial neural network, such as the weights of each connection between nodes.

For example, FIG. 8 includes a block diagram of an example of an analysis pipeline configured for training the ANN using data obtained from sequencing a reference sample. The ANN is configured to operate in the training mode. The sequencing instrument generates raw data files during a sequencing run on the reference sample using a targeted sequencing assay. For example, the assay may be the CarrierSeq assay (Thermo Fisher Scientific) or the Ampliseq Exome assay (Thermo Fisher Scientific). The raw data is provided to the signal processing module, which provides signal measurements for the reference sample. The input channels to the ANN during training mode, may be configured to be the same as those during inference mode. The base calling applied to the signal measurements for the reference sample generates predicted base calls and simulated signal measurements using a simulation model. The reference sequence corresponding to the reference sample is provided to the alignment module. Sequence reads output from the base caller are aligned with the reference sequence by the alignment module. The alignment identifies the labeled, or truth, base sequences that correspond to the sequence reads. The labeled base sequences are input to determine “truth” signal correction values. For example, the truth signal correction values can be the mean residual values derived from the difference between flow measurements and predicted flow measurements. The truth signal correction values are provided for training loss determination. The training loss values are used to update the parameters of the ANN.

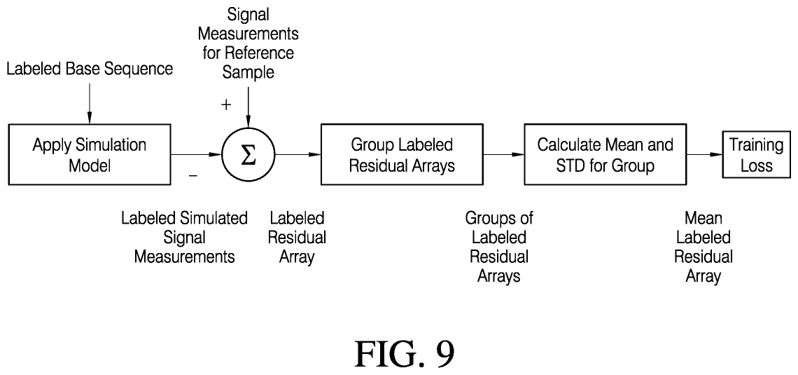

As illustrated in FIG. 9, the matched sequence and variants can be applied to a simulation model using model parameters. The simulated signal measurements can be used with the signal measurements for the reference sample to generate a residual array. The residual arrays from like sequences can be grouped and used to calculate mean and standard deviation to generate a mean residual array or variance array. When training the neural network, such a mean residual array or variance array can be used to determine training loss. Optionally, the ANN can provide a corrected flow measurement to be compared with the predicted flow measurement to provide additional training loss values. The training loss can be used to adjust the parameters of the neural network. The training optimizes the parameters of the ANN to minimize the mean squared error. The methods for training may comprise a machine learning algorithm, for example, a stochastic gradient descent (SGD) algorithm, Adam, Adadelta, Adagrad or other adaptive learning algorithm.

The artificial neural network architecture may be implemented in any processor. The network architecture may be implemented across multiple processors, multiple devices, or both.

According to various exemplary embodiments, one or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), graphics processing units (GPUs), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented using appropriately configured and/or programmed non-transitory machine-readable medium or article that may store an instruction or a set of instructions that, if executed by a machine, may cause the machine to perform a method and/or operations in accordance with the exemplary embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Hash memory, hard drive, tape, CDROM, etc.). Moreover, memory can incorporate electronic, magnetic, optical, and/or other types of storage media. Memory can have a distributed architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

According to various exemplary embodiments, one or more features of any one or more of the above-discussed teachings and/or exemplary embodiments may be performed or implemented at least partly using a distributed, clustered, remote, or cloud computing resource.

According to various exemplary embodiments, one or more features of any one or more of the above-discussed teachings and/or exemplary embodiments may be performed or implemented using a source program, executable program (object code), script, or any other entity comprising a set of instructions to be performed. When a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, R, Pascal, Basic, Fortran, Cobol, Perl, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

Example 1 is a method for correcting signal measurements, including: providing a plurality of signal measurements to a channel of an input layer to an artificial neural network (ANN), wherein the input layer includes one or more channels; applying the ANN to the plurality of signal measurements to generate a plurality of signal correction values; subtracting the plurality of signal correction values from the plurality of signal measurements to form a plurality of corrected signal measurements; and applying base calling to the plurality of corrected signal measurements to produce a sequence of base calls.

Example 2 includes the subject matter of Example 1, and further specifies that the input layer further includes a channel for a plurality of simulated signal measurements, wherein the plurality of simulated signal measurements corresponds to the plurality of signal measurements.

Example 3 includes the subject matter of Example 1, and further specifies that the input layer further includes a channel for representing a flow order corresponding to nucleotides flowed, wherein the plurality of signal measurements was detected in response to the nucleotides flowed in the flow order.

Example 4 includes the subject matter of Example 3, and further specifies that the flow order is represented by four binary arrays in four channels of the input layer, wherein a 1 in a position in the array indicates that a particular nucleotide was flowed in that position in the flow order to generate the corresponding signal measurement, wherein flow orders for nucleotides A, T, C, and G are each represented in a respective one of the arrays.

Example 5 includes the subject matter of Example 1, and further specifies that the input layer further includes a channel for an array of values indicating positions of the plurality of signal measurements.

Example 6 includes the subject matter of Example 1, and further specifies that the input layer further includes a channel for a signal mask, wherein the signal mask includes an array having 1's in positions corresponding to the plurality of signal measurements and 0's in positions corresponding to no signal measurement, wherein a number of signal measurements is less than or equal to a size of the array.

Example 7 includes the subject matter of Example 1, and further specifies that the ANN includes a convolutional neural network (CNN).

Example 8 includes the subject matter of Example 7, and further specifies that the CNN includes a U-Net.

Example 9 includes the subject matter of Example 8, and further specifies that the U-Net includes an encoder configured to receive the channels of the input layer and to generate feature maps at a plurality of scales.

Example 10 includes the subject matter of Example 9, and further specifies that the encoder further includes a plurality of layer groups, wherein each layer group includes one or more convolutional layers.

Example 11 includes the subject matter of Example 10, and further specifies that the convolutional layer applies a plurality of convolutions to input channels provided to the convolutional layer to produce a plurality of feature maps.

Example 12 includes the subject matter of Example 11, and further specifies that the convolutional layer further includes a batch normalization applied to the plurality of feature maps to produce normalized feature maps.

Example 13 includes the subject matter of Example 12, and further specifies that the convolutional layer further includes applying an activation function to the normalized feature maps to produce output feature maps for output channels of the convolutional layer.

Example 14 includes the subject matter of Example 13, and further specifies that the activation function includes a sigmoid linear unit function (SiLU).

Example 15 includes the subject matter of Example 10, and further specifies that each layer group further includes a pooling layer, wherein the pooling layer receives output channels from a last convolutional layer in the one or more convolutional layers.

Example 16 includes the subject matter of Example 15, and further specifies that the pooling layer applies a MaxPool operation having a kernel size of 2 and stride value of 2 to each output channel.

Example 17 includes the subject matter of Example 10, and further specifies that a number of the convolutional layers is two.

Example 18 includes the subject matter of Example 10, and further specifies that a number of layer groups in the plurality of layer groups is three.

Example 19 includes the subject matter of Example 9, and further specifies that the U-Net further includes a decoder, wherein the decoder receives the feature maps having the plurality of scales from the encoder.

Example 20 includes the subject matter of Example 19, and further specifies that the U-Net further includes a Convolutional Block Attention Module (CBAM), wherein the CBAM is applied to outputs of a last pooling layer of the encoder and provides refined feature maps to a first layer of the decoder.

Example 21 includes the subject matter of Example 19, and further specifies that the decoder further includes a second plurality of layer groups, wherein each layer group includes a convolution transpose layer.

Example 22 includes the subject matter of Example 21, and further specifies that the convolution transpose layer applies a plurality of transposed convolutions to input channels provided to the convolution transpose layer to produce a plurality of upsampled feature maps.

Example 23 includes the subject matter of Example 21, and further specifies that each layer group of the decoder further includes one or more convolutional layers.

Example 24 includes the subject matter of Example 23, and further specifies that the convolutional layer applies a plurality of convolutions to input channels provided to the convolutional layer to produce a plurality of feature maps.

Example 25 includes the subject matter of Example 24, and further specifies that the convolutional layer further includes a batch normalization applied to the plurality of feature maps to produce normalized feature maps.

Example 26 includes the subject matter of Example 25, and further specifies that the convolutional layer further includes applying an activation function to the normalized feature maps to produce output feature maps for output channels of the convolutional layer.

Example 27 includes the subject matter of Example 26, and further specifies that the activation function includes a sigmoid linear unit function (SiLU).

Example 28 includes the subject matter of Example 23, and further specifies that a number of the convolutional layers is two.

Example 29 includes the subject matter of Example 23, and further specifies that a first convolutional layer of the layer group of the decoder receives output channels from the convolution transpose layer of the layer group.

Example 30 includes the subject matter of Example 23, and further includes concatenating feature maps from a layer group of the encoder with feature maps from the convolution transpose layer to form concatenated feature maps, wherein the feature maps from the layer group of the encoder and the feature maps from the convolution transpose layer have a same scale.

Example 31 includes the subject matter of Example 30, and further includes applying a first convolutional layer of the layer group to the concatenated feature maps.

Example 32 includes the subject matter of Example 23, and further specifies that a second convolutional layer of the layer group of the decoder receives output channels from a first convolutional layer of the layer group.

Example 33 includes the subject matter of Example 21, and further specifies that a number of layer groups in the second plurality of layer groups is three.

Example 34 includes the subject matter of Example 21, and further includes applying a plurality convolutions to a plurality of outputs of a last layer group of the second plurality of layer groups to produce the plurality of signal correction values.

Example 35 includes the subject matter of Example 1, and further specifies that the plurality of signal measurements are provided by a nucleic acid sequencing instrument.

Example 36 is a system for correcting signal measurements, including: a machine-readable memory; and a processor configured to execute machine-readable instructions, which, when executed by the processor, cause the system to perform a method, including: providing a plurality of signal measurements to a channel of an input layer to an artificial neural network (ANN), wherein the input layer includes one or more channels; applying the ANN to the plurality of signal measurements to generate a plurality of signal correction values; subtracting the plurality of signal correction values from the plurality of signal measurements to form a plurality of corrected signal measurements; and applying base calling to the plurality of corrected signal measurements to produce a sequence of base calls.

Example 37 includes the subject matter of Example 36, and further specifies that the input layer further includes a channel for a plurality of simulated signal measurements, wherein the plurality of simulated signal measurements corresponds to the plurality of signal measurements.

Example 38 includes the subject matter of Example 36, and further specifies that the input layer further includes a channel for representing a flow order corresponding to nucleotides flowed, wherein the plurality of signal measurements was detected in response to the nucleotides flowed in the flow order.

Example 39 includes the subject matter of Example 38, and further specifies that the flow order is represented by four binary arrays in four channels of the input layer, wherein a 1 in a position in the array indicates that a particular nucleotide was flowed in that position in the flow order to generate the corresponding signal measurement, wherein flow orders for nucleotides A, T, C, and G are each represented in a respective one of the arrays.

Example 40 includes the subject matter of Example 36, and further specifies that the input layer further includes a channel for an array of values indicating positions of the plurality of signal measurements.

Example 41 includes the subject matter of Example 36, and further specifies that the input layer further includes a channel for a signal mask, wherein the signal mask includes an array having 1's in positions corresponding to the plurality of signal measurements and 0's in positions corresponding to no signal measurement, wherein a number of signal measurements is less than or equal to a size of the array.

Example 42 includes the subject matter of Example 36, and further specifies that the ANN includes a convolutional neural network (CNN).

Example 43 includes the subject matter of Example 42, and further specifies that the CNN includes a U-Net.

Example 44 includes the subject matter of Example 43, and further specifies that the U-Net includes an encoder configured to receive the channels of the input layer and to generate feature maps at a plurality of scales.

Example 45 includes the subject matter of Example 44, and further specifies that the encoder further includes a plurality of layer groups, wherein each layer group includes one or more convolutional layers.

Example 46 includes the subject matter of Example 45, and further specifies that the convolutional layer applies a plurality of convolutions to input channels provided to the convolutional layer to produce a plurality of feature maps.

Example 47 includes the subject matter of Example 46, and further specifies that the convolutional layer further includes a batch normalization applied to the plurality of feature maps to produce normalized feature maps.

Example 48 includes the subject matter of Example 47, and further specifies that the convolutional layer further includes applying an activation function to the normalized feature maps to produce output feature maps for output channels of the convolutional layer.

Example 49 includes the subject matter of Example 48, and further specifies that the activation function includes a sigmoid linear unit function (SiLU).

Example 50 includes the subject matter of Example 45, and further specifies that each layer group further includes a pooling layer, wherein the pooling layer receives output channels from a last convolutional layer in the one or more convolutional layers.

Example 51 includes the subject matter of Example 50, and further specifies that the pooling layer applies a MaxPool operation having a kernel size of 2 and stride value of 2 to each output channel.

Example 52 includes the subject matter of Example 45, and further specifies that a number of the convolutional layers is two.

Example 53 includes the subject matter of Example 45, and further specifies that a number of layer groups in the plurality of layer groups is three.

Example 54 includes the subject matter of Example 44, and further specifies that the U-Net further includes a decoder, wherein the decoder receives the feature maps having the plurality of scales from the encoder.

Example 55 includes the subject matter of Example 54, and further specifies that the U-Net further includes a Convolutional Block Attention Module (CBAM), wherein the CBAM is applied to outputs of a last pooling layer of the encoder and provides refined feature maps to a first layer of the decoder.

Example 56 includes the subject matter of Example 54, and further specifies that the decoder further includes a second plurality of layer groups, wherein each layer group includes a convolution transpose layer.

Example 57 includes the subject matter of Example 56, and further specifies that the convolution transpose layer applies a plurality of transposed convolutions to input channels provided to the convolution transpose layer to produce a plurality of upsampled feature maps.

Example 58 includes the subject matter of Example 56, and further specifies that each layer group of the decoder further includes one or more convolutional layers.

Example 59 includes the subject matter of Example 58, and further specifies that the convolutional layer applies a plurality of convolutions to input channels provided to the convolutional layer to produce a plurality of feature maps.

Example 60 includes the subject matter of Example 59, and further specifies that the convolutional layer further includes a batch normalization applied to the plurality of feature maps to produce normalized feature maps.

Example 61 includes the subject matter of Example 60, and further specifies that the convolutional layer further includes applying an activation function to the normalized feature maps to produce output feature maps for output channels of the convolutional layer.

Example 62 includes the subject matter of Example 61, and further specifies that the activation function includes a sigmoid linear unit function (SiLU).

Example 63 includes the subject matter of Example 58, and further specifies that a number of the convolutional layers is two.

Example 64 includes the subject matter of Example 58, and further specifies that a first convolutional layer of the layer group of the decoder receives output channels from the convolution transpose layer of the layer group.

Example 65 includes the subject matter of Example 58, and further includes concatenating feature maps from a layer group of the encoder with feature maps from the convolution transpose layer to form concatenated feature maps, wherein the feature maps from the layer group of the encoder and the feature maps from the convolution transpose layer have a same scale.

Example 66 includes the subject matter of Example 65, and further includes applying a first convolutional layer of the layer group to the concatenated feature maps.

Example 67 includes the subject matter of Example 58, and further specifies that a second convolutional layer of the layer group of the decoder receives output channels from a first convolutional layer of the layer group.

Example 68 includes the subject matter of Example 56, and further specifies that a number of layer groups in the second plurality of layer groups is three.

Example 69 includes the subject matter of Example 56, and further includes applying a plurality convolutions to a plurality of outputs of a last layer group of the second plurality of layer groups to produce the plurality of signal correction values.

Example 70 includes the subject matter of Example 36, and further specifies that the plurality of signal measurements are provided by a nucleic acid sequencing instrument.

Example 71 is a non-transitory machine-readable storage medium including instructions which, when executed by a processor, cause the processor to perform a method for correcting signal measurements, including: providing a plurality of signal measurements to a channel of an input layer to an artificial neural network (ANN), wherein the input layer includes one or more channels; applying the ANN to the plurality of signal measurements to generate a plurality of signal correction values; subtracting the plurality of signal correction values from the plurality of signal measurements to form a plurality of corrected signal measurements; and applying base calling to the plurality of corrected signal measurements to produce a sequence of base calls.

Example 72 includes the subject matter of Example 71, and further specifies that the input layer further includes a channel for a plurality of simulated signal measurements, wherein the plurality of simulated signal measurements corresponds to the plurality of signal measurements.

Example 73 includes the subject matter of Example 71, and further specifies that the input layer further includes a channel for representing a flow order corresponding to nucleotides flowed, wherein the plurality of signal measurements was detected in response to the nucleotides flowed in the flow order.

Example 74 includes the subject matter of Example 71, and further specifies that the flow order is represented by four binary arrays in four channels of the input layer, wherein a 1 in a position in the array indicates that a particular nucleotide was flowed in that position in the flow order to generate the corresponding signal measurement, wherein flow orders for nucleotides A, T, C, and G are each represented in a respective one of the arrays.

Example 75 includes the subject matter of Example 71, and further specifies that the input layer further includes a channel for an array of values indicating positions of the plurality of signal measurements.

Example 76 includes the subject matter of Example 71, and further specifies that the input layer further includes a channel for a signal mask, wherein the signal mask includes an array having 1's in positions corresponding to the plurality of signal measurements and 0's in positions corresponding to no signal measurement, wherein a number of signal measurements is less than or equal to a size of the array.

Example 77 includes the subject matter of Example 71, and further specifies that the ANN includes a convolutional neural network (CNN).

Example 78 includes the subject matter of Example 77, and further specifies that the CNN includes a U-Net.

Example 79 includes the subject matter of Example 78, and further specifies that the U-Net includes an encoder configured to receive the channels of the input layer and to generate feature maps at a plurality of scales.

Example 80 includes the subject matter of Example 79, and further specifies that the encoder further includes a plurality of layer groups, wherein each layer group includes one or more convolutional layers.

Example 81 includes the subject matter of Example 80, and further specifies that the convolutional layer applies a plurality of convolutions to input channels provided to the convolutional layer to produce a plurality of feature maps.

Example 82 includes the subject matter of Example 81, and further specifies that the convolutional layer further includes a batch normalization applied to the plurality of feature maps to produce normalized feature maps.

Example 83 includes the subject matter of Example 82, and further specifies that the convolutional layer further includes applying an activation function to the normalized feature maps to produce output feature maps for output channels of the convolutional layer.

Example 84 includes the subject matter of Example 83, and further specifies that the activation function includes a sigmoid linear unit function (SiLU).

Example 85 includes the subject matter of Example 80, and further specifies that each layer group further includes a pooling layer, wherein the pooling layer receives output channels from a last convolutional layer in the one or more convolutional layers.

Example 86 includes the subject matter of Example 85, and further specifies that the pooling layer applies a MaxPool operation having a kernel size of 2 and stride value of 2 to each output channel.

Example 87 includes the subject matter of Example 80, and further specifies that a number of the convolutional layers is two.

Example 88 includes the subject matter of Example 80, and further specifies that a number of layer groups in the plurality of layer groups is three.

Example 89 includes the subject matter of Example 79, and further specifies that the U-Net further includes a decoder, wherein the decoder receives the feature maps having the plurality of scales from the encoder.

Example 90 includes the subject matter of Example 89, and further specifies that the U-Net further includes a Convolutional Block Attention Module (CBAM), wherein the CBAM is applied to outputs of a last pooling layer of the encoder and provides refined feature maps to a first layer of the decoder.

Example 91 includes the subject matter of Example 89, and further specifies that the decoder further includes a second plurality of layer groups, wherein each layer group includes a convolution transpose layer.

Example 92 includes the subject matter of Example 91, and further specifies that the convolution transpose layer applies a plurality of transposed convolutions to input channels provided to the convolution transpose layer to produce a plurality of upsampled feature maps.

Example 93 includes the subject matter of Example 91, and further specifies that each layer group of the decoder further includes one or more convolutional layers.

Example 94 includes the subject matter of Example 93, and further specifies that the convolutional layer applies a plurality of convolutions to input channels provided to the convolutional layer to produce a plurality of feature maps.

Example 95 includes the subject matter of Example 94, and further specifies that the convolutional layer further includes a batch normalization applied to the plurality of feature maps to produce normalized feature maps.

Example 96 includes the subject matter of Example 95, and further specifies that the convolutional layer further includes applying an activation function to the normalized feature maps to produce output feature maps for output channels of the convolutional layer.

Example 97 includes the subject matter of Example 96, and further specifies that the activation function includes a sigmoid linear unit function (SiLU).

Example 98 includes the subject matter of Example 93, and further specifies that a number of the convolutional layers is two.

Example 99 includes the subject matter of Example 93, and further specifies that a first convolutional layer of the layer group of the decoder receives output channels from the convolution transpose layer of the layer group.

Example 100 includes the subject matter of Example 93, and further includes concatenating feature maps from a layer group of the encoder with feature maps from the convolution transpose layer to form concatenated feature maps, wherein the feature maps from the layer group of the encoder and the feature maps from the convolution transpose layer have a same scale.

Example 101 includes the subject matter of Example 100, and further includes applying a first convolutional layer of the layer group to the concatenated feature maps.

Example 102 includes the subject matter of Example 93, and further specifies that a second convolutional layer of the layer group of the decoder receives output channels from a first convolutional layer of the layer group.

Example 103 includes the subject matter of Example 91, and further specifies that a number of layer groups in the second plurality of layer groups is three.

Example 104 includes the subject matter of Example 91, and further includes applying a plurality convolutions to a plurality of outputs of a last layer group of the second plurality of layer groups to produce the plurality of signal correction values.

Example 105 includes the subject matter of Example 71, and further specifies that the plurality of signal measurements are provided by a nucleic acid sequencing instrument.

What is claimed is:

1. A method for correcting signal measurements, comprising:
providing a plurality of signal measurements to a channel of an input layer to an artificial neural network (ANN), wherein the input layer includes one or more channels;
applying the ANN to the plurality of signal measurements to generate a plurality of signal correction values;
subtracting the plurality of signal correction values from the plurality of signal measurements to form a plurality of corrected signal measurements; and
applying base calling to the plurality of corrected signal measurements to produce a sequence of base calls.

2. The method of claim 1, wherein the input layer further comprises a channel for a plurality of simulated signal measurements, wherein the plurality of simulated signal measurements corresponds to the plurality of signal measurements.

3. The method of claim 1, wherein the input layer further comprises a channel for representing a flow order corresponding to nucleotides flowed, wherein the plurality of signal measurements was detected in response to the nucleotides flowed in the flow order.

4. The method of claim 1, wherein the ANN comprises a convolutional neural network (CNN).

5. The method of claim 4, wherein the CNN comprises a U-Net.

6. The method of claim 5, wherein the U-Net comprises an encoder configured to receive the channels of the input layer and to generate feature maps at a plurality of scales.

7. The method of claim 6, wherein the encoder further comprises a plurality of layer groups, wherein each layer group comprises one or more convolutional layers, wherein the convolutional layer applies a plurality of convolutions to input channels provided to the convolutional layer to produce a plurality of feature maps.

8. The method of claim 7, wherein the convolutional layer further includes a batch normalization applied to the plurality of feature maps to produce normalized feature maps.

9. The method of claim 8, wherein the convolutional layer further includes applying an activation function to the normalized feature maps to produce output feature maps for output channels of the convolutional layer.

10. The method of claim 7, wherein each layer group further comprises a pooling layer, wherein the pooling layer receives output channels from a last convolutional layer in the one or more convolutional layers, wherein the pooling layer applies a MaxPool operation.

11. The method of claim 6, wherein the U-Net further comprises a decoder, wherein the decoder receives the feature maps having the plurality of scales from the encoder.

12. The method of claim 11, wherein the U-Net further comprises a Convolutional Block Attention Module (CBAM), wherein the CBAM is applied to outputs of a last pooling layer of the encoder and provides refined feature maps to a first layer of the decoder.

13. The method of claim 11, wherein the decoder further comprises a second plurality of layer groups, wherein each layer group comprises a convolution transpose layer.

14. The method of claim 13, wherein the convolution transpose layer applies a plurality of transposed convolutions to input channels provided to the convolution transpose layer to produce a plurality of upsampled feature maps.

15. The method of claim 13, wherein each layer group of the decoder further comprises one or more convolutional layers, wherein the convolutional layer applies a plurality of convolutions to input channels provided to the convolutional layer to produce a plurality of feature maps.

16. The method of claim 15, wherein the convolutional layer further includes a batch normalization applied to the plurality of feature maps to produce normalized feature maps.

17. The method of claim 16, wherein the convolutional layer further includes applying an activation function to the normalized feature maps to produce output feature maps for output channels of the convolutional layer.

18. The method of claim 15, wherein a first convolutional layer of the layer group of the decoder receives output channels from the convolution transpose layer of the layer group, further comprising:
concatenating feature maps from a layer group of the encoder with feature maps from the convolution transpose layer to form concatenated feature maps, wherein the feature maps from the layer group of the encoder and the feature maps from the convolution transpose layer have a same scale; and
applying the first convolutional layer of the layer group to the concatenated feature maps.

19. The method of claim 15, wherein a second convolutional layer of the layer group of the decoder receives output channels from a first convolutional layer of the layer group.

20. The method of claim 13, further comprising applying a plurality convolutions to a plurality of outputs of a last layer group of the second plurality of layer groups to produce the plurality of signal correction values.
